(19) United States
(12) Patent Application Publication
(10) Pub. No.: US 2015/0183839 A1
(43) Pub. Date: Jul. 2, 2015

(54) SOYBEAN NODULATION REGULATORY PEPTIDES AND METHODS OF USE

(71) Applicants: Peter M. Gresshoff, Indooroopilly (AU); Brett Ferguson, St. Lucia (AU); Dugald Reid, Kangaroo Point (AU)

(72) Inventors: Peter M. Gresshoff, Indooroopilly (AU); Brett Ferguson, St. Lucia (AU); Dugald Reid, Kangaroo Point (AU)

(21) Appl. No.: 13/807,676

(22) PCT Filed: Jun. 30, 2011
(86) PCT No.: PCT/AU2011/000818

§ 371 Date: Aug. 29, 2013

Related U.S. Application Data
(60) Provisional application No. 61/398,854, filed on Jun. 30, 2010.

(51) Int. Cl. C07K 14/415; C12N 15/82; C07K 16/16

(52) U.S. Cl. CPC C07K 14/415; C07K 16/16; C12N 15/8262

(57) ABSTRACT

Peptide fragments of CLAVATA3/ESR-related (CLE) proteins of soybean are provided which are activators of autoregulation of nodulation in soybean. CLE30 and CLE80 peptide fragments may act systemically while CLE60 peptides may act locally in the plant root. CLE30 and CLE80 are normally induced in response to Rhizobium inoculation of the plant root, while CLE60 acts as a nitrate or ammonium sensor. Also provided are interfering mutant CLE peptides and proteins, encoding nucleic acids and constructs for genetic modification of leguminous plants as a means for control of NARK-dependent nodulation and/or nitrogen fixation, particularly with regard to major leguminous crop plants such as soybean and common bean.

### TECHNICAL FIELD

THIS INVENTION relates to nodulation and/or nitrogen fixation in legumes. More particularly, this invention relates to proteins that are components of a mechanism in legumes for controlling nodulation and/or nitrogen fixation and to uses of these proteins or encoding nucleic acids in modulating nodulation and/or nitrogen fixation in plants.

### BACKGROUND

Many legume species can form a symbiotic relationship with nitrogen fixing soil bacteria collectively called rhizobia. This occurs via a complex signalling exchange between the plant and bacteria and results in the formation of specialised root organs known as nodules (Ferguson et al. 2010). Within the nodule, the plant receives fixed atmospheric nitrogen from the bacteria in exchange for photoassimilates. This symbiotic nitrogen fixation is critical to legume cultivation as it provides both economic and environmental advantages over other crops. Additionally, the development of nodules provides an excellent system to study lateral organ development and regulation in plants.

Nodulation commences following the plant's perception of specialised signal molecules produced by the bacteria called Nod factors (Ferguson and Mathesius 2003; Ferguson et al. 2010). Soybean (Glycine max) is the most widely cultivated legume and forms determinate type nodules in a symbiosis with Bradyrhizobium japonicum and Rhizobium fredii. In soybean, Nod factors are perceived by the receptors, NFR5 (Nod Factor Receptor 5; Indrasumunar et al., 2010) and NFR1 (International Publication WO2007/070960). Downstream signalling events in the root lead to the re-initiation of cortical cell divisions and the initiation of a lateral meristem.

Recently, these early signalling events have been relatively well studied in model legume species, including Lotus japonicus, Medicago truncatula, Pisum sativum and G. max. This has allowed for the identification of several key components required for successful nodule development (see reviews by Ferguson et al., 2010; Oldroyd & Downie, 2008). Initial signalling events lead to calcium spiking in and surrounding the nucleus and trigger the activation of CCamK (e.g., Kosuta et al., 2008; Oldroyd & Downie, 2006), which together with the activation of a cytokinin receptor (e.g., Murray et al., 2007; Tirichine et al., 2007) results in the modulation of the expression of several early nodulation genes (e.g., Heckmann et al., 2006; Schauser et al., 1999; Vernie et al., 2008).

Nodule development requires a large investment of energy by the plant and balanced allocation of resources to growing points. Therefore, the plant regulates the number of nodules it forms in order to optimise its need to acquire nitrogen with its ability to expend energy. This occurs in response to both internal and external stimuli, including to pre-existing infection events and to environmentally available nitrogen. The inbuilt mechanism that plants use to regulate their nodule number is called the Autoregulation Of Nodulation (AON). This mechanism is responsible for legume plants exhibiting a distinct crown nodulation phenotype where nodules develop predominately in the zone of nodulation. This zone is the region of the root that at the time of inoculation has emerging root hairs that are susceptible to rhizobia-infection (Bhuvaneswari et al., 1980; Bhuvaneswari et al., 1981; Calvert et al., 1984).

The current model for AON (Ferguson et al., 2010; Magori & Kawaguchi, 2009) begins with the production of a root-derived cue (Q) that is thought to be produced following both the first nodulation-induced cell divisions in the root (Caetano-Anollés & Gresshoff, 1990; Li et al., 2009) and at the onset of nitrogen fixation (Li et al., 2009). Q is subsequently transported to the shoot, likely via the xylem stream, where it is perceived by an LRR receptor kinase. This LRR receptor kinase is structurally, but not functionally similar to CLAVATA1 from Arabidopsis, and is encoded by GmNARK in soybean (Searle et al., 2003) and its orthologues in other legumes (MtSUNN, Schnabel et al., 2005; LjHAR1. Nishimura et al., 2002; PsSYM29, Krusell et al., 2002). To date, GmNARK (and its orthologs LjHAR1, PsSYM29, MtSUNN) is the only genetic component that has been unequivocally identified in the AON pathway (Searle et al., 2003), although a kinase-associated protein phosphatase (KAPP) was shown to interact with GmNARK (Miyahara et al., 2008).

Using grafting and split-root studies, it has been shown to act systemically in the shoot to regulate nodule numbers in the root (Delves et al., 1986; Delves et al., 1987; Olsson et al., 1989). Following the perception of Q by NARK, a novel signal is produced that acts as a shoot-derived inhibitor (SDI) of nodulation. SDI is transported from the shoot to the roots where it prevents further nodulation events. Work to identify SDI has so far indicated that it is likely a small molecule that is not a protein or RNA (Lin et al., 2010).

Mutants defective in the negative regulator AON loop lack the ability to regulate their nodule numbers and therefore form a super- or hyper-nodulation phenotype (Carroll et al., 1985a; b). In soybean, all AON mutants isolated have been found to have a mutation in GmNARK (Searle et al. 2003). These AON mutants are also nitrate tolerant, as they form nodules in the presence of nitrate levels that are otherwise inhibitory to nodulation in wild-type plants (Carroll et al., 1985a; b). That GmNARK mutations affect both AON and nitrate regulation of nodulation indicates that this gene is a component of both of these regulatory mechanisms. This would also appear to explain why GmNARK expression is observed in the phloem parenchyma of both the shoot and the root (Nontachaiyapoom et al., 2007). Furthermore, recent work has shown that both local and systemic regulatory mechanisms of nodulation are occurring in response to nitrate (Jeudy et al., 2010). To date, the molecular components of NARK-dependent regulation of nodulation in soybean and other crop legumes remain unknown.

### SUMMARY

The invention is broadly directed to protein regulators of nodulation in legumes, particularly crop legumes. In one particular form, the protein regulators are particular CLAVATA3/ESR-related (CLE) proteins of crop legumes such as soybean.

In a first aspect, the invention provides an isolated protein which comprises, consists essentially of or consists of an amino acid sequence corresponding to a fragment of a CLAVATA3/ESR-related (CLE) protein that is responsive to Rhizobiales inoculation or nitrate or ammonium in a leguminous plant.

Suitably, the leguminous plant is a crop plant.

Preferably the leguminous crop plant is Glycine max.

In particular embodiments, the isolated protein comprises, consists of or consists essentially of an amino acid sequence set forth in SEQ ID NO:1, SEQ ID NO:2 or SEQ ID NO:3.

The peptides of SEQ ID NOS:1-3 are characterised by the consensus sequence RLX₁P₁X₂GP₂DX₃X₄HX₅ (SEQ ID NO:4) wherein X₁=serine or alanine; X₂=glycine or glutamate; X₃=proline or asparagine; X₄=histidine, lysine or glutamine; and X₅=histidine or asparagine, with the proviso that when X₁ is serine, X₅ is histidine.

Preferably, the CLE protein is selected from the group consisting of “CLE30” (SEQ ID NO:5; also referred to herein as “RIC1”), “CLE60” (SEQ ID NO:6; also referred to herein as “NIC1”) and “CLE80” (SEQ ID NO:7; also referred to herein as “RIC2”)

Accordingly in one embodiment the isolated protein of this aspect comprises an amino acid sequence set forth in SEQ ID NO:5, SEQ ID NO:6, or SEQ ID NO:7.

This aspect also includes fragments, variants and derivatives of said isolated protein.

In one embodiment, the variant comprises one or more amino acid deletions or substitutions in RLX₁P₁X₂GP₂DX₃X₄HX₅ (SEQ ID NO:4) selected from the group consisting of (i) R is absent or is substituted by another amino acid; (ii) X₁ is absent or is substituted by an amino acid other than A or S; (iii) P₁ is absent or is substituted by another amino acid; (iv) P₂ is absent or is substituted by another amino acid; (v) G is absent or substituted by an amino acid other than alanine; (vi) D is absent or is substituted by another amino acid; (vii) H is absent or is substituted by another amino acid; and (viii) X₅ is absent or is substituted by an amino acid other than H or N.

In a second aspect, the invention provides an antibody or antibody fragment which binds and/or is raised against the isolated protein of the first aspect.

The antibody may be a monoclonal antibody or a polyclonal antibody.

In one embodiment, the antibody is a recombinant antibody.

In a third aspect, the invention provides an isolated nucleic acid comprising a nucleotide sequence that encodes the isolated protein of the first aspect or the recombinant antibody of the second aspect, or a nucleotide sequence complementary thereto.

In particular embodiments, the isolated nucleic acid comprises a nucleotide sequence set forth in any one of SEQ ID NOS: 8-10.

In another embodiment the isolated nucleic acid of this aspect comprises a nucleotide sequence set forth in SEQ ID NO:11, SEQ ID NO:12, or SEQ ID NO:13.

This aspect also includes fragments and variants of said isolated nucleic acid.

Isolated nucleic acids also include a promoter or promoter active fragment of a gene encoding the isolated protein of the first aspect.

In particular embodiments, the promoter or promoter-active fragment comprises a nucleotide sequence set forth in any one of SEQ ID NOS: 14-16.

In a fourth aspect, the invention provides a genetic construct comprising (i) the isolated nucleic acid of the third aspect; or (ii) an isolated nucleic acid comprising a nucleotide sequence complementary thereto; operably linked or connected to one or more regulatory sequences in an expression vector.

In a fifth aspect, the invention provides a genetically-modified leguminous plant, or one or more cells or tissue of said plant, comprising the isolated nucleic acid of the second aspect or the genetic construct of the third aspect.

Preferably, the genetically-modified leguminous plant, one or more cells of tissues displays relatively improved, enhanced and/or otherwise facilitated nodulation and/or nitrogen fixation.

In a sixth aspect, the invention provides a method of producing a genetically-modified leguminous plant, cell or tissue including the step of introducing the isolated nucleic acid of the second aspect or the genetic construct of the third aspect into a cell or tissue of a leguminous plant to thereby genetically-modify said plant cell or tissue.

Preferably, the method produces a genetically-modified leguminous plant, cell or tissue that displays relatively improved, enhanced and/or otherwise facilitated nodulation and/or nitrogen fixation.

In a seventh aspect, the invention provides a method of breeding a leguminous plant, said method including the step of crossing parent leguminous plants to produce a progeny leguminous plant having a desired trait associated with CLE protein-regulated, wherein at least one of the parent leguminous plants is selected as having the desired trait.

Preferably, the method of breeding produces a leguminous plant that displays relatively improved, enhanced and/or otherwise facilitated nodulation and/or nitrogen fixation.

Suitably, the leguminous plant is a crop plant.

Preferably the leguminous crop plant is soybean (Glycine max) or common bean (Phaseolus vulgaris).

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE FIGURES

FIG. 1. Micro-synteny and homology between GmCLE genes. Amino acid conservation (% identity) between each of the CLE genes was highest between each gene (white) and its inactive duplicate (hashed) and between the inoculation responsive CLE's [A]. Phytozome cluster analysis showed a well-conserved region of synteny exists between species for CLE30/CLE80 [B] and CLE60 [C].

FIG. 2. Multiple sequence alignment of legume inoculation and nitrate responsive CLEs [A]. The predicted signal peptide of the soybean CLE peptides is underlined in black, the conserved signal peptide residues altered in CLE60 are enclosed by a box and the conserved 12 amino acid CLE domain is enclosed by a box closest to the C-terminus. SEQ ID NO:1=amino acid sequence of CLE 30 12 mer peptide; SEQ ID NO:2=amino acid sequence of CLE 60 12 mer peptide; and SEQ ID NO:3=amino acid sequence of CLE 80 12 mer peptide. SEQ ID NO:5=full length CLE30 amino acid sequence; SEQ ID NO:6=full length CLE60 amino acid sequence; and SEQ ID NO:7=full length CLE80 amino acid sequence. [B]. A logo alignment (Crooks et al., 2004) of the CLE domain and C-terminal extension illustrates the degree of conservation at each residue

FIG. 3. Gene expression of CLE30 and CLE60 in the zone of nodulation after rhizobia inoculation [A]. Plants were inoculated with compatible (WT) or incompatible (nodC-) rhizobia before gene expression was quantified using RT-qPCR. CLE30 was significantly increased as early as 12 h after inoculation with WT bacteria whereas CLE60 was only slightly induced and was not detectable until later. Gene expression of CLE60 in the root after nitrate treatment was significantly increased as early as 8 h after treatment and increased further at 24 h [B]. Error bars indicate SE.

FIG. 4. CLE expression relative to nitrate concentration and nodule numbers. Tissue samples were harvested 2 wks following inoculation and 3 wks following nitrate treatment. CLE60 expression was significantly induced by 2 mM nitrate and plateaued at 5 mM. CLE80 expression level correlated strongly with nodule numbers which were significantly reduced as nitrate concentration increased. CLE30 expression level did not appear to correlate with nodulation at this later stage of development. Error bars indicate SE.

FIG. 5. Effect of transgenic overexpression of inoculation-induced CLE genes on nodule number. Transgenic hairy-roots induced with the vector-only control showed a normal nodulation phenotype on Williams WT plants whereas nodules were eliminated in WT roots over-expressing CLE30 or CLE80. In contrast, nodule numbers were not reduced in nod4 mutant plants, mutated in the GmNARK receptor kinase gene. Error bars indicate SE.

FIG. 6. Effect of transgenic overexpression of nitrate-induced CLE gene CLE60 on nodule number. Transgenic hairy-roots induced with the vector-only control showed a normal nodulation phenotype on Williams WT plants whereas nodules were reduced or partially eliminated in WT roots over-expressing CLE60. In contrast, nodule numbers were not reduced in nod4 mutant plants, mutated in the GmNARK receptor kinase gene. Suppression of nodulation as significant but not complete, caused by the presence of roots failing to express the CLE60 gene. Error bars indicate SE.

FIG. 7. Grafting of Williams (WT) scions with nod4 root-stocks under different nitrate conditions. Grafted plants having WT root-stock had significantly fewer nodules when treated with either 5 mM or 10 mM nitrate compared with those having nod4 root-stocks or those not treated with nitrate. Error bars indicate SE.

FIG. 8. Proposed model of NARK-dependent CLE activity in the root and shoot. AON involves long-distance signalling requiring the interaction of CLE30 or CLE80 with NARK in the leaf phloem parenchyma of the vascular system and the subsequent inhibition of nodulation via the production of a Shoot-Derived Inhibitor (SDI). Local nitrate inhibition of nodulation is established by the interaction of CLE60 with NARK in the root leading to production of a SDI-like Nitrate Induced Inhibitor (NII) of nodulation.

FIG. 9. CLE expression relative to nitrate concentration. Tissue samples were harvested after 2 wks nitrate treatment (no Bradyrhizobium inoculation). CLE60 expression was significantly induced by 10 mM nitrate relative to untreated plants (0 mM) whereas CLE30 was unaltered between treatment groups. Error bars indicate SE.

FIG. 10. (A) CLE30, CLE60 and CLE80 nucleotide sequences. Nucleotide sequences encoding the 12 mer CLE peptides are underlined. SEQ ID NO:8=nucleotide sequence encoding CLE30 12 mer; SEQ ID NO:9=nucleotide sequence encoding CLE60 12 mer; SEQ ID NO:10=nucleotide sequence encoding CLE80 12 mer; SEQ ID NO:11=nucleotide sequence encoding full length CLE30 protein; SEQ ID NO:12=nucleotide sequence encoding full length CLE60 protein; SEQ ID NO:13=nucleotide sequence encoding full length CLE80 protein. (B) CLE30, CLE60 and CLE80 full length protein sequences. SEQ ID NO:5=amino acid sequence of CLE30; SEQ ID NO:6=amino acid sequence of CLE60; and SEQ ID NO:7=amino acid sequence of CLE80.

FIG. 11. (A) CLE30 and CLE60 promoter nucleotide sequence; and (B) CLE80 promoter nucleotide sequence. Each promoter sequence terminates in an ATG initiation codon for the prepropeptide. SEQ ID NO:14=nucleotide sequence of CLE30 gene promoter; SEQ ID NO:15=nucleotide sequence of CLE60 gene promoter; and SEQ ID NO:16=nucleotide sequence of CLE80 gene promoter.

FIG. 12. CLE30 (GmRIC1) expression in G. max inhibits nodulation.

FIG. 13. CLE30 (GmRIC1) expression in of G. soja inhibits nodulation.

FIG. 14. CLE30 (GmRIC1) expression in WT P. vulgaris inhibits nodulation and has no effect when expressed in the AON mutant R32.

FIG. 15. To test if CLE60 (GmNIC1) is induced by alternative nitrogen sources we treated soybeans with 10 mM solutions of the following chemicals for two weeks prior to harvesting root tissue to analyse gene expression: Ammonium Chloride (NH₄Cl), Potassium Nitrate (KNO₃) and Potassium Chloride control (KCl).

### DETAILED DESCRIPTION

The invention is at least partly predicated on the discovery that CLE30, CLE60 and CLE80 proteins are activators of autoregulation of nodulation (AON) in leguminous crop plants such as soybean (Glycine max: the world's major legume crop) and common bean (Phaseolus vulgaris: the world's humanly most consumed legume). More particularly, these CLE proteins may act systemically in the case of CLE30 and CLE80 or may act locally in the plant root, in the case of CLE60. A further discovery is that CLE30 and CLE80 are normally induced in response to Bradyrhizobium inoculation of the plant root, while CLE60 acts as a “nitrogen status sensor”, such as by acting as a “nitrate sensor” or an “ammonium sensor”. While not wishing to be bound by any particular theory, it is proposed that nitrate induces CLE60 promoter activity to thereby increase CLE60 protein expression to thereby influence NARK-dependent NII (nitrate induced inhibition). Identification of the CLE proteins of the invention therefore provides a unique means for control of NARK-dependent nodulation and/or nitrogen fixation, particularly with regard to major leguminous crop plants of importance in countries such as USA, Brazil, China, Argentina, and India, although without limitation thereto. A particularly preferred embodiment of the invention provides genetically-modified leguminous crop plants that displays relatively improved, enhanced and/or otherwise facilitated nodulation and/or nitrogen fixation.

As used herein, “leguminous crop plants” include soybean species of the Glycine genus such as Glycine max and Glycine soja, alfalfa, clovers, beans (e.g., Phaseolus beans, azukibeans, Faba beans), lentils, acacia (wattle) species, garden pea, pulses (cowpea, pigeonpea, chickpea), Pongamia, lupins, mesquite, and peanuts, although without limitation thereto. It will be appreciated that data provided hereinafter in the Examples show that CLE proteins function to regulate nitrogen fixation in soybean (Glycine max), ancestral soybean (Glycine soja), common bean (Phaseolus vulgaris), pea (Pisum sativum) and Lotus (Lotus japonicus). Of particular note is that a CLE peptide originating from soybean suppresses nodulation in common bean (Phaseolus vulgaris).

In one aspect, the invention provides an isolated protein which comprises, consists of or consists essentially of an amino acid sequence of a fragment of a CLAVATA3/ESR-related (CLE) protein that is responsive to Rhizobiales inoculation or nitrate in a leguminous crop plant.

The term “Rhizobiales” includes and encompasses members of the order of nitrogen-fixing bacterial order Rhizobiales such as Sinorhizobium, Bradyrhizobium, Mesorhizobium and Rhizobium, although without limitation thereto.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form.

By “protein” is also meant an amino acid polymer, comprising natural and/or non-natural amino acids, including L- and D-isomeric forms as are well-understood in the art. In this context, the standard IUPAC single letter amino acid code will be used in some cases to indicate amino acid residues.

A “peptide” is a protein having no more than sixty (60) contiguous amino acids.

A “polypeptide” is a protein having more than sixty (60) contiguous amino acids.

As described herein, in one embodiment the invention provides “full length” CLE proteins in the form of CLE30 (RIC1), CLE80 (RIC2) and CLE60 (NIC1) proteins of soybean comprising an amino acid sequence set forth in SEQ ID NOS:5-7, respectively.

It will be appreciated that in another form, the isolated protein of the invention is a peptide fragment of a CLE protein that comprises, consists of or consists essentially of at least twelve (12) contiguous amino acids of a CLE protein that mediates NARK-dependent AON and NII. Suitably, the isolated protein of this form is not a full length CLE protein.

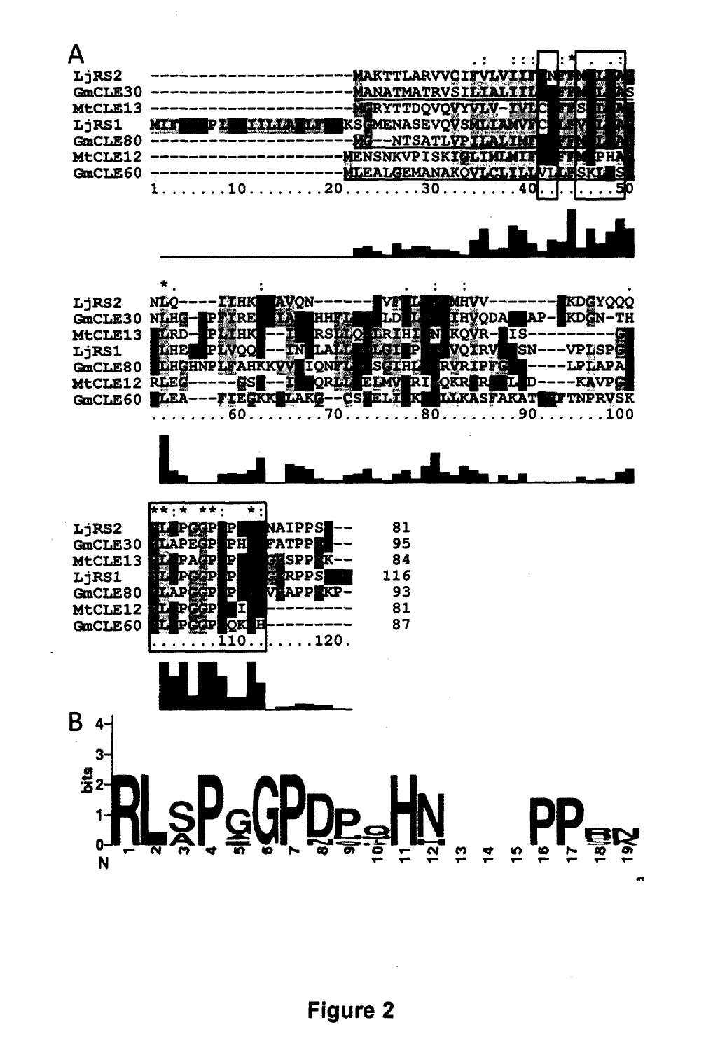

As shown in FIG. 2, the CLE30, CLE60 and CLE80 proteins of Glycine max respectively comprise “CLE peptide” domains or motifs characterised by the consensus sequence RLX₁P₁X₂GP₂DX₃X₄HX₅ (SEQ ID NO:4) wherein X₁=serine or alanine; X₂=glycine or glutamate; X₃=proline or asparagine; X₄=histidine, lysine or glutamine; and X₅=histidine or asparagine, with the proviso that when X₁ is serine, X₅ is histidine.

In particular embodiments, the isolated protein comprises, consists essentially of, or consists of an amino acid sequence set forth in SEQ ID NO:1, SEQ ID NO:2 or SEQ ID NO:3.

In this context, by “consists essentially of” means that the isolated protein comprises the amino acid sequence of any one of SEQ ID NOS:1-4 together with 1 or 2 additional amino acids at the N- or C-terminus.

Although not wishing to be bound by any theory, it is proposed that CLE proteins or shorter CLE peptides comprising as few as the twelve (12) amino acids of SEQ ID NOS:5-7, defined by SEQ ID NO:4 or more specifically SEQ ID NOS:1-3, may be sufficient to function in NARK-dependent AON and/or NII. In addition, such CLE peptides may further comprise one or more signal peptide amino acids and/or one or more conserved C-terminal amino acid residues as shown in FIG. 2.

Furthermore, site-directed mutagenesis to convert each amino acid in SEQ ID NO:4 into an alanine (other than A in X₁) followed by over-expression of the mutant protein in transgenic soybean hairy-roots has identified modifications that resulted in reduced CLE activity (i.e. reduced suppression of nodulation). The underlined amino acids of SEQ ID NO:4 reduce suppression when replaced with an alanine: RLX₁P₁X₂GP₂DX₃X₄HX₅.

This aspect of the invention also includes fragments, variants and derivatives of said isolated protein, whether a CLE peptide “fragment” or a “full length” CLE protein disclosed herein.

In one embodiment, a protein “fragment” includes an amino acid sequence which constitutes less than 100%, but at least 20%, preferably at least 30%, more preferably at least 80% or even more preferably at least 90%, 95%, 96%, 97%, 98% or 99% of an amino acid sequence of an isolated protein of the invention or of a full length CLE protein.

In another embodiment, a “fragment” is a peptide, for example of at least 6, preferably at least 10 and more preferably at least 12, 15, 20, 25, 30, 35, 40, 45, 50 or 55 amino acids in length. Larger fragments or polypeptides comprising more than one peptide are also contemplated, and may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard, which is included in a publication entitled “Synthetic Vaccines” edited by Nicholson and published by Blackwell Scientific Publications. Alternatively, peptides can be produced by digestion of a protein of the invention with suitable proteases. The digested fragments can be purified by, for example, by high performance liquid chromatographic (HPLC) techniques.

Suitably, the fragment is a “biologically active fragment” which retains biological activity of said isolated protein or said full length CLE protein.

The biologically active fragment of the isolated protein preferably has greater than 10%, preferably greater than 20%, more preferably greater than 50% and even more preferably greater than 75%, 80%, 85%, 90% 95%, 96%, 97%, 98% or 99% of the biological activity of the entire protein.

Another example of a biologically-active fragment is a fragment of CLE30, CLE60 or CLE80 lacking the N-terminal signal peptide shown in FIG. 2.

As used herein, a “variant” protein is an isolated protein of the invention in which one or more amino acids have been deleted or substituted by different amino acids, or other amino acids added to the amino acid sequence. These substitutions may be conservative or non-conservative.

Variants include naturally occurring (e.g., allelic) variants, orthologs (i.e., from species other than Glycine max) and synthetic variants, such as produced in vitro using mutagenesis techniques.

In one embodiment, a variant protein comprises one or more amino acid deletions or substitutions of the underlined amino acids in SEQ ID NO:4: RLX₁PX₂GPDX₃X₄HX₁.

In a particularly preferred embodiment, the one or more amino acid substitutions are non-conservative substitutions (e.g. substituted by alanine), whereby [symbol] substituted by an amino acid other than alanine; (vi) D is absent or is substituted by another amino acid; (vii) H is absent or is substituted by another amino acid; and (viii) X₅ is absent or is substituted by an amino acid other than H or N.

It will be appreciated that the one or more amino acid deletions or substitutions may be made in any of the CLE peptide sequences of SEQ ID NOS:1-3 (as characterized by the consensus sequence of SEQ ID NO:4) or in any of the full length CLE protein sequences of SEQ ID NOS:5-7.

Preferably, orthologs and paralogs are obtainable from other leguminous crop plants such as alfalfa, clovers, French beans, azukibeans, Faba beans, lentils, garden pea, cowpea, pigeonpea, mungbean, chickpea, lupins, mesquite, carob and peanuts, although without limitation thereto.

It will be appreciated that in certain embodiments, amino acid sequence variations may be conservative, resulting in variants that essentially retain the biological activity of a corresponding CLE protein or peptide (e.g., allelic variants, paralogs and orthologs). In certain other embodiments, amino acid sequence variations may be non-conservative, resulting in variants that lack, or have a substantially reduced, biological activity compared to a corresponding CLE protein or peptide.

In some embodiments, variants have an amino acid sequence at least 75%, 80%, 85%, 90% or 95%, 96%, 97%, 98% or 99% amino acid sequence identity to a CLE peptide, such as set forth in SEQ ID NOS:1-4, or an isolated protein comprising an amino acid sequence set forth in SEQ ID NOS:5-7.

Terms used herein to describe sequence relationships between respective nucleic acids and proteins include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/polypeptides, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically at least 6, 8, 10 or 12 contiguous residues that is compared to a reference sequence. The comparison window may comprise additions or deletions (i.e., gaps) of about 20% or less as compared to the reference sequence (which does not comprise additions or deletions) for optimal alignment of the respective sequences. Optimal alignment of sequences for aligning a comparison window may be conducted by computerised implementations of algorithms (for example ECLUSTALW and BESTFIT provided by WebAngis GCG, 2D Angis, GCG and GeneDoc programs) or by inspection and the best alignment (i.e., resulting in the highest percentage similarity or identity over the comparison window) generated by any of the various methods selected.

The ECLUSTALW program can be used to align multiple sequences. This program calculates a multiple alignment of nucleotide or amino acid sequences according to a method by Thompson, J. D., Higgins, D. G. and Gibson, T. J. (1994). This is part of the original ClustalW distribution, modified for inclusion in EGCG. The BESTFIT program aligns forward and reverse sequences and sequence repeats. This program makes an optimal alignment of a best segment of similarity between two sequences. Optimal alignments are determined by inserting gaps to maximize the number of matches using the local homology algorithm of Smith and Waterman. ECLUSTALW and BESTFIT alignment packages are offered in WebANGIS GCG (The Australian Genomic Information Centre, Building JO3, The University of Sydney, N.S.W 2006, Australia).

Reference also may be made to the BLAST family of programs as for example disclosed by Altschul et al., 1997, Nucl. Acids Res. 25 3389.

A detailed discussion of sequence analysis can be found in Chapter 19.3 of Ausubel et al, supra.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical amino acid (or nucleotide base in the case of nucleic acids) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software Engineering Co., Ltd., South San Francisco, Calif., USA).

With regard to protein variants, these can be created by mutagenising a protein or an encoding nucleic acid, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra.

It will be appreciated by the skilled person that site-directed mutagenesis is best performed where knowledge of the amino acid residues that contribute to biological activity is available.

In cases where this information is not available, or can only be inferred by molecular modeling approximations, for example, random mutagenesis is contemplated. Random mutagenesis methods include chemical modification of proteins by hydroxylamine (Ruan et al., 1997, Gene 188 35), incorporation of dNTP analogs into nucleic acids (Zaccolo et al., 1996, J. Mol. Biol. 255 589) and PCR-based random mutagenesis such as described in Stemmer, 1994, Proc. Natl. Acad. Sci. USA 91 10747 or Shafikhani et at, 1997, Biotechniques 23 304. It is also noted that PCR-based random mutagenesis kits are commercially available, such as the Diversify™ kit (Clontech).

Mutagenesis may also be induced by chemical means, such as ethyl methane sulphonate (EMS) and/or irradiation means, such as fast neutron irradiation of seeds as known in the art and in particular relation to soybean (Carroll et al, 1985, Proc. Natl. Acad. Sci. USA 82 4162; Carroll et al, 1985, Plant Physiol. 78 34; Men et al., 2002, Genome Letters 3 147).

As used herein, “derivative” proteins are proteins of the invention that have been altered, for example by conjugation or complexing with other chemical moieties or by post-translational modification as would be understood in the art. Such derivatives include amino acid substitutions, deletions and/or additions to proteins and peptides of the invention, or variants thereof. Protein derivatives may also include modifications such as glycosylation, partial or complete de-glycosylation, phosphorylation, acetylation, lipid- or de-lipidation, alkylation, amidation, nitrosylation, sulfation, sulfhydryl reduction, ubiquitination and removal of signal peptides, although without limitation thereto.

“Additions” of amino acids may include fusion of the peptide or polypeptides of the invention, or variants thereof, with other peptides or polypeptides. Particular examples of such peptides include amino (N) and carboxyl (C) terminal amino acids added for use as fusion partners or “tags”.

Well-known examples of fusion partners include hexahistidine (6X-HIS)-tag, N-Flag, Fc portion of human IgG, glutathione-S-transferase (GST) and maltose binding protein (MBP), which are particularly useful for isolation of the fusion polypeptide by affinity chromatography. For the purposes of fusion polypeptide purification by affinity chromatography, relevant matrices for affinity chromatography may include nickel-conjugated or cobalt-conjugated resins, fusion polypeptide specific antibodies, glutathione-conjugated resins, and amylose-conjugated resins respectively. Some matrices are available in “kit” form, such as the ProBond™ Purification System (Invitrogen Corp.) which incorporates a 6X-His fusion vector and purification using ProBond™ resin.

The fusion partners may also have protease cleavage sites, for example enterokinase (available from Invitrogen Corp. as EnterokinaseMax™), Factor Xₐ or Thrombin, which allow the relevant protease to digest the fusion polypeptide of the invention and thereby liberate the recombinant polypeptide of the invention therefrom. The liberated polypeptide can then be isolated from the fusion partner by subsequent chromatographic separation.

Fusion partners may also include within their scope “epitope tags”, which are usually short peptide sequences for which a specific antibody is available.

Other derivatives contemplated by the invention include, chemical modification to side chains, incorporation of unnatural amino acids and/or their derivatives during peptide or polypeptide synthesis and the use of cross linkers and other methods which impose conformational constraints on the polypeptides, fragments and variants of the invention.

Non-limiting examples of side chain modifications contemplated by the present invention include chemical modifications of amino groups, carboxyl groups, guanidine groups of arginine residues, sulphydryl groups, tryptophan residues, tyrosine residues and/or the imidazole ring of histidine residues, as are well understood in the art.

Non-limiting examples of incorporating unnatural amino acids and derivatives during peptide synthesis include, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

Isolated proteins of the invention may be produced by recombinant DNA technology or by chemical synthesis, as are well known in the art.

Recombinant proteins may be produced, as for example described in Sambrook, et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. 1995-2009), in particular Chapters 10 and 16; and CURRENT PROTOCOLS IN PROTEIN SCIENCE Eds. Coligan et al., (John Wiley & Sons, Inc. 1995-2009), in particular Chapters 1, 5, 6 and 7.

In a further aspect, the invention provides an antibody or antibody fragment which binds or is raised against a CLE peptide motif or domain according to SEQ ID NOS:1-4, a CLE protein according to SEQ ID NOS:5-7 or a fragment variant or derivative, as hereinbefore described. In one embodiment, the antibody is an inhibitory or blocking antibody which at least partly prevents, inhibits or blocks a CLE protein activity, such as binding of a CLE protein or peptide to a NARK receptor.

Antibodies may be polyclonal or monoclonal. Antibodies also include recombinant antibodies and antibody fragments, as are well understood in the art.

Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988.

Generally, antibodies of the invention bind to or conjugate with a CLE peptide, as hereinbefore described. For example, the antibodies may comprise polyclonal antibodies. Such antibodies may be prepared for example by injecting a CLE peptide or a protein comprising the CLE peptide into a production species, which may include mice, rabbits or goats, to obtain polyclonal antisera. Methods of producing polyclonal antibodies are well known to those skilled in the art. Exemplary protocols that may be used are described for example in Coligan et al., CURRENT PROTOCOLS IN IMMUNOLOGY, supra, and in Harlow & Lane, 1988, supra.

Monoclonal antibodies may be produced using the standard method as for example, described in an article by Köhler & Milstein, 1975, Nature 256, 495, or by more recent modifications thereof as for example, described in Coligan et al., CURRENT PROTOCOLS IN IMMUNOLOGY, supra by immortalizing spleen or other antibody producing cells derived from a production species which has been inoculated with a CLE peptide or a protein comprising the CLE peptide.

The invention also includes within its scope antibodies that comprise antibody fragments such as Fc, Fab of F(ab)′2 fragments of the polyclonal, monoclonal or recombinant antibodies referred to above. Alternatively, the antibodies may comprise single chain Fv antibodies (scFvs) against CLE peptides of the invention.

In another aspect, the invention provides an isolated nucleic acid that comprises a nucleotide sequence that encodes an isolated CLE peptide or protein of the invention, or a nucleotide sequence complementary thereto. In particular embodiments, the isolated nucleic acid comprises or consists of a nucleotide sequence set forth in SEQ ID NOS:8-13. In other embodiments, the isolated nucleic acid comprises a promoter or promoter-active fragment of a CLE gene, such as set forth in SEQ ID NOS:14-16.

The term “nucleic acid” as used herein designates single- or double-stranded DNA or RNA and DNA:RNA hybrids. DNA includes cDNA and genomic DNA. RNA includes mRNA, cRNA, interfering RNA such as RNAi, siRNA and catalytic RNA such as ribozymes. A nucleic acid may be native or recombinant and may comprise one or more artificial nucleotides, e.g., nucleotides not normally found in nature. Nucleic acids may include modified purines (for example, inosine, methylinosine and methyladenosine) and modified pyrimidines (thiouridine and methylcytosine).

The terms “mRNA”, “RNA” and “transcript” are used interchangeably when referring to a transcribed copy of a nucleic acid.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labelled for the purpose of detecting complementary sequences in Northern blotting, Southern blotting or microarray analysis, for example.

A “primer” is usually a single-stranded oligonucleotide, preferably having 20-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

The invention also contemplates fragments of isolated nucleic acids of the invention such as may be useful for recombinant protein expression or as probes, primers and the like.

Another embodiment of a nucleic acid fragment is a “promoter-active fragment” which is capable of initiating, directing, controlling or otherwise facilitating RNA transcription. With particular reference to SEQ ID NOS:14-16, a promoter active fragment may comprise at least 100, 200, 300, 400, 500, 600, 700, 800, 900, 1000 or 2000 contiguous nucleotides of the promoter sequence.

As used herein, the term “variant”, in relation to an isolated nucleic acid, includes naturally-occurring allelic variants.

Variants also include nucleic acids that have been mutagenised or otherwise altered so as to encode a protein having the same amino acid sequence (e.g., through degeneracy), or a modified amino acid sequence. These alterations may include deletion, substitution or addition of one or more nucleotides in a promoter. The alteration may either increase or decrease activity as required. In this regard, nucleic acid mutagenesis may be performed in a random fashion or by site-directed mutagenesis in a more “rational” manner. Standard mutagenesis techniques are well known in the art, and examples are provided in Chapter 9 of CURRENT PROTOCOLS IN MOLECULAR BIOLOGY Eds Ausubel et al. (John Wiley & Sons NY, 1995). Mutagenesis also includes mutagenesis using chemical and/or irradiation methods such as EMS and fast neutron mutagenesis of plant seeds.

In another embodiment, nucleic acid variant are nucleic acids having one or more codon sequences altered by taking advantage of codon sequence redundancy. A particular example of this embodiment is optimization of a nucleic acid sequence according to codon usage as is well known in the art. This can effectively “tailor” a nucleic acid for optimal expression in a particular organism, or cells thereof, where preferential codon usage has been established.

Nucleic acid variants also include within their scope “homologs”, “orthologs” and “paralogs”.

Nucleic acid orthologs may be isolated, derived or otherwise obtained from plants other than Glycine max. Preferably, orthologs are obtainable from leguminous crop plants such alfalfa, clovers, beans such as Phaseolus beans, azukibeans and Faba beans, lentils, peas such as cowpea, pigeonpea and chickpea, lupins, mesquite, carob and peanuts, although without limitation thereto.

In another embodiment, nucleic acid homologs share at least 80%, preferably at least 85%, more preferably at least 90%, 95%, 96%, 97%, 98% or 99%, sequence identity with a nucleotide sequence encoding an isolated CLE peptide or protein of the invention, such as set forth in SEQ ID NOS:8-13 or the promoter sequences of SEQ ID NOS:14-16.

In yet another embodiment, nucleic acid homologs hybridise to nucleotide sequence encoding a CLE peptide of the invention.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

Modified purines (for example, inosine, methylinosine and methyladenosine) and modified pyrimidines (thiouridine and methylcytosine) may also engage in base pairing.

“Stringency” as used herein, refers to temperature and ionic strength conditions, and presence or absence of certain organic solvents and/or detergents during hybridisation. The higher the stringency, the higher will be the required level of complementarity between hybridising nucleotide sequences.

“Stringent conditions” designates those conditions under which only nucleic acid having a high frequency of complementary bases will hybridize.

Reference herein to high stringency conditions include and encompasses:—

(i) from at least about 31% v/v to at least about 50% v/v formamide and from at least about 0.01 M to at least about 0.15 M NaCl for hybridisation at 42° C., and at least about 0.01 M to at least about 0.15 M salt for washing at 42° C.;

(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and

(iii) 0.2×SSC, 0.1% SDS for washing at or above 68° C. for about 20 minutes.

Notwithstanding the above, stringent conditions are well-known in the art, such as described in Chapters 2.9 and 2.10 of Ausubel et al., supra. A skilled addressee will also recognise that various factors can be manipulated to optimise the specificity of the hybridisation. Optimisation of the stringency of the final washes can serve to ensure a high degree of hybridisation.

Typically, complementary nucleotide sequences are identified by blotting techniques that include a step whereby nucleotides are immobilised on a matrix (preferably a synthetic membrane such as nitrocellulose), a hybridisation step, and a detection step.

In light of the foregoing, it will be appreciated that variants, homologs and orthologs may also be isolated by means such as nucleic acid sequence amplification techniques, (including but not limited to PCR, strand displacement amplification, rolling circle amplification, helicase-dependent amplification and the like) and techniques which employ nucleic acid hybridisation (e.g., plaque/colony hybridisation).

In yet another particular aspect, the invention provides a genetic construct. The genetic construct may preferably comprise (i) an isolated nucleic acid comprising a nucleotide sequence encoding an isolated CLE peptide (such as according to SEQ ID NOS:8-10); (ii) an isolated nucleic acid comprising a nucleotide sequence encoding an entire CLE30, CLE60 and/or a CLE80 protein (such as according to SEQ ID NOS:11-13; (iii) or a nucleotide sequence complementary to (i) or (ii). The genetic construct may encode a recombinant antibody or antibody fragment, such as an inhibitory recombinant antibody or antibody fragment.

Accordingly, the invention provides a genetic construct that may comprise a nucleic acid that encodes a fragment of a protein CLE protein (e.g., a CLE peptide) or a full length CLE30, CLE60 and/or CLE80 protein. In other embodiments, the genetic construct may comprise a promoter or promoter-active fragment of a CLE30, CL60 and/or CLE80 gene, such as according to any one of SEQ ID NOS:14-16. According to one particular form of this embodiment, a CLE60 promoter may be inducible, or otherwise “sensitive” to nitrate and/or changes in levels of nitrate. Thus, genetic constructs comprising the CLE60 promoter may be useful in nitrate-dependent expression of CLE60 peptides and/or heterologous nucleic acids encoding other proteins.

As used herein, a “genetic construct” further comprises one or more regulatory elements that facilitate manipulation, propagation, homologous recombination and/or expression of said nucleic acid.

In a preferred form, the genetic construct is an expression construct comprising an expression vector.

In one form, the genetic construct is suitable for the expression of an isolated nucleic acid of the invention that encodes one or more CLE proteins or peptides in leguminous crop plants.

In another form, the genetic construct is suitable for reduction, inhibition or down-regulation of CLE protein and/or nucleic acid expression in leguminous crop plants.

For example, the genetic construct may be suitable for inactivation or “knock out” of an endogenous CLE30, CLE60 and/or CLE80 gene in a leguminous crop plant.

An expression construct may comprise or express an inhibitory nucleic acid (e.g., for expressing an inhibitory RNA) such as siRNA, RNAi, ribozymeor anti-sense RNA constructs that facilitate down-regulation of CLE protein expression in leguminous crop plants. Another example of an expression construct that facilitates down-regulation of CLE protein expression in leguminous crop plants encodes an inhibitory recombinant antibody or antibody fragment that binds a CLE peptide.

In another embodiment, the expression construct encodes an interfering mutant CLE peptide or protein.

In one particular embodiment, an interfering mutant CLE protein or peptide is, or comprises, one or more non-conservative amino acid substitutions of the underlined amino acids in SEQ ID NO:4: RLX₁PX₂GPDX₃X₄HX₅, as hereinbefore described. For example, one ore more amino acid substitutions may be introduced into a CLE protein amino acid sequence set forth in any of SEQ ID NOS:5-7 or into a 12 mer peptide amino acid sequence according to any of SEQ ID NOS:1-3.

Typically, an expression construct comprises one or more regulatory sequences present in an expression vector, operably linked or operably connected to the nucleic acid of the invention, to thereby assist, control or otherwise facilitate transcription and/or translation of the isolated nucleic acid of the invention.

By “operably linked” or “operably connected” is meant that said regulatory nucleotide sequence(s) is/are positioned relative to the nucleic acid or chimeric gene of the invention to initiate, regulate or otherwise control transcription and/or translation.

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, promoter sequences, leader or signal sequences, ribosomal binding sites, transcriptional start and termination sequences, translational start and termination sequences, and enhancer or activator sequences.

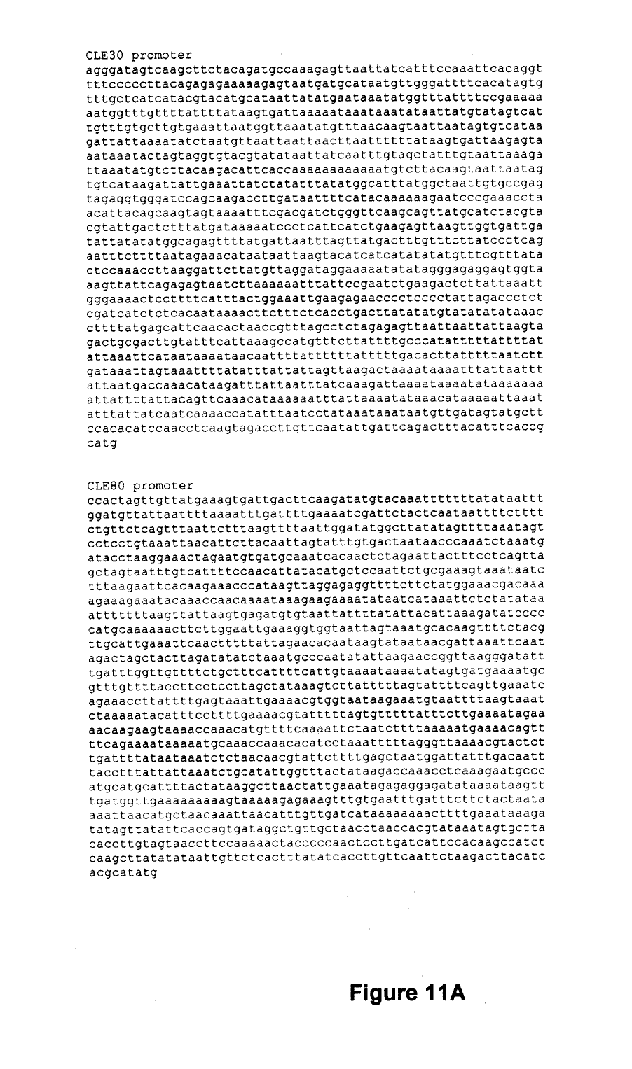

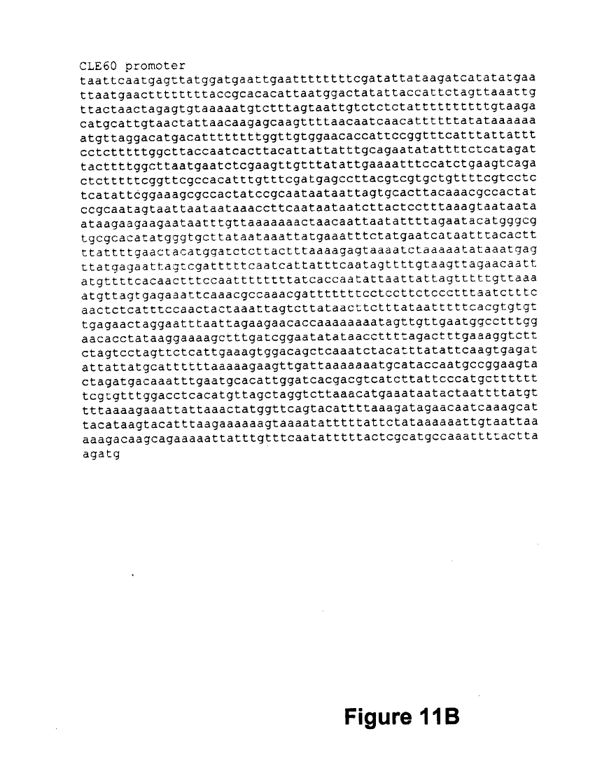

In embodiments, the promoter may be an autologous promoter. For example, a promoter-active fragment of a corresponding CLE gene (e.g., CLE30, CLE60 or CLE80) nucleic acid may effectively act as an autologous promoter. Non-limiting examples of such autologous promoters are provided in FIG. 11 (SEQ ID NOS:14-16).

In alternative embodiments the expression construct may comprise a heterologous promoter operable in a leguminous crop plant.

Non-limiting examples of suitable heterologous promoters include the CaMV35S promoter, Emu promoter (Last et al., 1991, Theor. Appl. Genet. 81 581) or the maize ubiquitin promoter Ubi (Christensen & Quail, 1996, Transgenic Research 5 213).

A preferred heterologous promoter is the CaMV35S promoter.

Usually, when transgenic expression of a protein is required, a correct orientation of the encoding nucleic acid is in the sense or 5′ to 3′ direction relative to the promoter. However, where antisense expression is required, the nucleic acid is oriented 3′ to 5′. Both possibilities are contemplated by the expression construct of the present invention, and directional cloning for these purposes may be assisted by the presence of a polylinker.

An expression construct may further comprise viral and/or plant pathogen nucleotide sequences. A plant pathogen nucleic acid includes T-DNA plasmid, modified (including for example a recombinant nucleic acid) or otherwise, from Agrobacterium.

The expression construct may further comprise a selectable marker nucleic acid to allow the selection of transformed cells.

In embodiments relating to expression in plants, suitable selection markers include, but are not limited to, neomycin phosphotransferase II which confers kanamycin and geneticin/G418 resistance (nptII; Raynaerts et al., In: Plant Molecular Biology Manual A9:1-16. Gelvin & Schilperoort Eds (Kluwer, Dordrecht, 1988), bialophos/phosphinothricin resistance (bar; Thompson et al., 1987, EMBO J. 6 1589), streptomycin resistance (aadA; Jones et at, 1987, Mol. Gen. Genet. 210 86) paromomycin resistance (Mauro et al., 1995, Plant Sci. 112 97), β-glucuronidase (gus; Vancanneyt et al., 1990, Mol. Gen. Genet. 220 245) and hygromycin resistance (hmr or hpt; Waldron et al., 1985, Plant Mol. Biol. 5 103; Perl et al., 1996, Nature Biotechnol. 14 624).

Selection markers such as described above may facilitate selection of transformed plant cells or tissue by addition of an appropriate selection agent post-transformation, or by allowing detection of plant tissue which expresses the selection marker by an appropriate assay. In that regard, a reporter gene such as gfp, nptII, luc or gusA may function as a selection marker.

Positive selection is also contemplated such as by the phosphomannine isomerase (PMI) system described by Wang et al., 2000, Plant Cell Rep. 19 654 and Wright et al., 2001, Plant Cell Rep. 20 429 or by the system described by Endo et al., 2001, Plant Cell Rep. 20 60, for example.

The expression construct of the present invention may also comprise other gene regulatory elements, such as a 3′ non-translated sequence. A 3′ non-translated sequence refers to that portion of a gene that contains a polyadenylation signal and any other regulatory signals capable of effecting mRNA processing or gene expression. The polyadenylation signal is characterised by effecting the addition of polyadenylic acid tracts to the 3′ end of the mRNA precursor. Polyadenylation signals are commonly recognised by the presence of homology to the canonical form 5′ AATAAA-3′ although variations are not uncommon.

The 3′ non-translated regulatory DNA sequence preferably includes from about 300 to 1,000 nucleotide base pairs and contains plant transcriptional and translational termination sequences. Examples of suitable 3′ non-translated sequences are the 3′ transcribed non-translated regions containing a polyadenylation signal from the nopaline synthase (nos) gene of Agrobacterium tumefaciens (Bevan et al., 1983, Nucl. Acid Res., 11 369) and the terminator for the T7 transcript from the octopine synthase (ocs) gene of Agrobacterium tumefaciens.

Transcriptional enhancer elements include elements from the CaMV 35S promoter and octopine synthase (ocs) genes, as for example described in U.S. Pat. No. 5,290,924. It is proposed that the use of an enhancer element such as the ocs element, and particularly multiple copies of the element, may act to increase the level of transcription from adjacent promoters when applied in the context of plant transformation.

Additionally, targeting sequences may be employed to target an expressed protein to an intracellular compartment within plant cells or to the extracellular environment. For example, a DNA sequence encoding a transit or signal peptide sequence may be operably linked to a sequence encoding a desired protein such that, when translated, the transit or signal peptide can transport the protein to a particular intracellular or extracellular destination, respectively, and can then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane. For example, the transit or signal peptide can direct a desired protein to a particular organelle such as a plastid (e.g., a chloroplast), rather than to the cytoplasm. Thus, the expression construct can further comprise a plastid transit peptide encoding DNA sequence operably linked between a promoter region or promoter variant according to the invention and transcribable nucleic acid. For example, reference may be made to Heijne et al., 1989, Eur. J. Biochem. 180 535 and Keegstra et al., 1989, Ann. Rev. Plant Physiol. Plant Mol. Biol. 40 471.

A genetic construct may also include an element(s) that permits stable integration of the construct into the host cell genome or autonomous replication of the vector in the cell independent of the genome of the cell. The vector may be integrated into the host cell genome when introduced into a host cell. For integration, the vector may rely on the foreign or endogenous DNA sequence or any other element of the vector for stable integration of the vector into the genome by homologous recombination. Alternatively, the vector may contain additional nucleic acid sequences for directing integration by homologous recombination into the genome of the host cell. The additional nucleic acid sequences enable the vector to be integrated into the host cell genome at a precise location in the chromosome. To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences.

In other embodiments, a genetic construct may be used for expressing an isolated protein of the invention, or full length CLE protein, in a bacterial cell (e.g., E. coli DH5a or BL21), such as for recombinant protein production, an inducible promoter may be utilised, such as the IPTG-inducible lacZ promoter.

Other regulatory elements that may assist recombinant protein expression in bacteria include bacterial origins of replication (e.g., as in plasmids pBR322, pUC19 and the ColE1 replicon which function in many E. coli strains) and bacterial selection marker genes (amp^{r}, tet^{r} and kan^{r}, for example). It will also be appreciated that genetic constructs for plant expression may comprise one or more of the above regulatory elements to facilitate propagation in bacteria.

The genetic construct, whether for expression in plant or bacterial cells, may also include a fusion partner (typically provided by the expression vector) so that a recombinant protein is expressed as a fusion protein with the fusion partner, as hereinbefore described. An advantage of fusion partners is that they assist identification and/or purification of the fusion protein. Identification and/or purification may include using a monoclonal antibody or substrate specific for the fusion partner.

The invention also provides a genetically-modified plant, cell or tissue including comprising: (i) an isolated nucleic acid comprising a nucleotide sequence encoding an isolated protein of the invention in the form of a CLE peptide (such as according to any one of SEQ ID NOS:8-10); (ii) an isolated nucleic acid comprising a nucleotide sequence encoding a full length CLE protein (such as according to SEQ ID NOS:11-13); (iii) a nucleotide sequence complementary to (i) or (ii); or (iv) the genetic construct as hereinbefore described.

Also provided is a method of producing a genetically-modified plant, cell or tissue including the step of introducing: (i) an isolated nucleic acid comprising a nucleotide sequence encoding an isolated protein in the form of a CLE peptide (such as according to any one of SEQ ID NOS:8-10); (ii) an isolated nucleic acid comprising a nucleotide sequence encoding a full length CLE protein (such as according to any one of SEQ ID NOS:11-13); (iii) a nucleotide sequence complementary to (i) or (ii); or (iv) the genetic construct as hereinbefore described, into a cell or tissue of a leguminous crop plant to thereby genetically-modify said plant cell or tissue.

In one form, the isolated nucleic acid of the invention encodes one or more CLE peptides or full-length CLE proteins, to thereby “over-express” the one or more CLE peptides or full length proteins in the genetically-modified leguminous crop plant. This embodiment may be particularly advantageous for at least partly suppressing, reducing or inhibiting nodulation in the genetically-modified leguminous crop plant. This may improve the ability of the genetically-modified leguminous crop plant to absorb water and nutrients from soil. Such plants may have increased water and nutrient absorption thereby improving crop yields. For example, reduced nodulation may inhibit or suppress lateral root formation, thereby improving drought tolerance in the genetically-modified leguminous crop plant.

Alternatively, in a preferred embodiment the genetically-modified plant displays down-regulated or at least partly inhibited CLE protein expression or activity to thereby block or suppress CLE-mediated AON in the genetically-modified non-leguminous crop plant. In some embodiments, siRNA, RNAi, ribozyme or anti-sense RNA constructs may facilitate down-regulation of CLE protein expression to thereby block or suppress CLE-mediated AON in the genetically-modified non-leguminous crop plant.

In another embodiment, a nucleic acid or genetic construct may be introduced that encodes an interfering mutant CLE protein or peptide or an antibody that binds a CLE peptide to thereby block or suppress CLE-mediated AON in the genetically-modified leguminous crop plant.

In one embodiment, an interfering mutant CLE protein or peptide is, or comprises, one or more non-conservative amino acid substitutions of the underlined amino acids in SEQ ID NO:4: RLX₁PX₂GPDX₃X₄HX₅, as hereinbefore described. For example, one ore more amino acid substitutions may be introduced into a CLE protein amino acid sequence set forth in any of SEQ ID NOS:5-7 or into a 12 mer peptide amino acid sequence according to any of SEQ ID NOS:1-3.

These embodiments may be particularly advantageous for inducing or promoting hyper- or super-nodulation in the genetically-modified plant. Enhanced or increased nodulation (e.g., super- or hypernodulation) can increase nitrogen fixation. Genetically-modified plants made in accordance with the present invention may be engineered to increase nodulation and nitrogen fixation in leguminous crop plants, thereby decreasing a requirement for nitrogen fertilisers. Enhanced or increased nodulation may also be useful when using nodules as bio-factories to produce a desired compound, such as a bio-active compound or biologically active protein. Increasing the number and/or frequency of nodules may improve yield and ease of harvesting of the bio-active compound that may be recombinantly expressed or endogenous to the nodule and/or symbiotic organism of the nodule.

It will be appreciated that “relatively” increased or reduced nodulation and/or nitrogen fixation is typically determined by comparison of nodulation and/or nitrogen fixation in a plant without genetic modification, preferably of the same plant species.

It will be appreciated that one particular advantage provided by the present invention is that a skilled person may select which one or more CLE proteins or peptides defined by the amino acid sequences of SEQ ID NOS:1-7, or mutants thereof, to over-express or inhibit, as required. As will be described in more detail in the Examples, the CLE proteins or peptides comprising the amino acid sequences of SEQ ID NOS: 1, 3, 5 and 8 act systemically to mediate control of nodulation in response to Rhizobiales inoculation. However, there are differences in expression pattern and timing that may be exploited. Furthermore, the CLE peptide of SEQ ID NOS:2 or full length CLE protein of SEQ ID NO:7 is uniquely involved locally in the plant root as a :nitrogen status sensor” (e.g a “nitrate sensor” or “ammonium sensor”). Thus, this CLE peptide pathway could be targeted to selectively modulate nodulation in response to nitrate.

Accordingly, the invention provides an opportunity to selectively over-express or inhibit any one or more of CLE30, CLE60 or CLE80 in a leguminous plant depending on a desired outcome.

In one embodiment, the method of producing a genetically-modified leguminous plant, plant cell or tissue, includes the steps of
(i) transforming a plant cell or tissue obtained from a leguminous crop plant with an expression construct as hereinbefore described; and
(ii) selectively propagating a genetically-modified plant from the plant cell or tissue transformed in step (i).

Suitably, the plant cell or tissue used at step (i) may be a leaf disk, callus, meristem, hypocotyls, root, leaf spindle or whorl, leaf blade, stem, shoot, petiole, axillary bud, shoot apex, internode, cotyledonary-node, flower stalk or inflorescence tissue.

Preferably, the plant tissue is a leaf or part thereof, including a leaf disk, hypocotyl or cotyledonary-node.

Persons skilled in the art will be aware that a variety of transformation methods are applicable to the method of the invention, such as Agrobacterium tumefaciens-mediated (Gartland & Davey, 1995, Agrobacterium Protocols (Humana Press Inc. NJ USA); U.S. Pat. No. 6,037,522; WO99/36637), microprojectile bombardment (Franks & Birch, 1991, Aust. J. Plant. Physiol., 18 471; Bower et al., 1996, Molecular Breeding, 2 239; Nutt et al., 1999, Proc. Aust. Soc. SugarCane Technol. 21 171), liposome-mediated (Ahokas et al., 1987, Heriditas 106 129), laser-mediated (Guo et al., 1995, Physiologia Plantarum 93 19), silicon carbide or tungsten whiskers (U.S. Pat. No. 5,302,523; Kaeppler et al., 1992, Theor. Appl. Genet. 84 560), virus-mediated (Brisson et al., 1987, Nature 310 511), polyethylene-glycol-mediated (Paszkowski et al., 1984, EMBO J. 3 2717) as well as transformation by microinjection (Neuhaus et al., 1987, Theor. Appl. Genet. 75 30) and electroporation of protoplasts (Fromm et al., 1986, Nature 319 791).

Agrobacterium-mediated transformation may utilise A. tumefaciens or A. rhizogenes.

Preferably, selective propagation at step (ii) is performed in a selection medium comprising geneticin as selection agent.

In one embodiment, the expression construct may further comprise a selection marker nucleic acid as hereinbefore described.

In another embodiment, a separate selection construct may be included at step (i), which selection construct comprises a selection marker nucleic acid.

The transformed plant material may be cultured in shoot induction medium followed by shoot elongation media as is well known in the art. Shoots may be cut and inserted into root induction media to induce root formation as is known in the art.

It will be appreciated that as discussed hereinbefore, there are a number of different selection agents useful according to the invention, the choice of selection agent being determined by the selection marker nucleic acid used in the expression construct or provided by a separate selection construct.

The “transgenic” status of genetically-modified plants of the invention may be ascertained by measuring expression of a CLE protein, peptide or encoding nucleic acid.

In one embodiment, transgene expression can be detected by an antibody specific for a CLE peptide:
(i) in an ELISA such as described in Chapter 11.2 of CURRENT PROTOCOLS IN MOLECULAR BIOLOGY Eds. Ausubel et al, (John Wiley & Sons Inc. NY, 1995); or
(ii) by Western blotting and/or immunoprecipitation such as described in Chapter 12 of CURRENT PROTOCOLS IN PROTEIN SCIENCE Eds. Coligan et al. (John Wiley & Sons Inc. NY, 1997).

Protein-based techniques such as mentioned above may also be found in Chapter 4.2 of PLANT MOLECULAR BIOLOGY: A Laboratory Manual, supra.

It will also be appreciated that genetically-modified plants of the invention may be screened for the presence of mRNA encoding a CLE peptide nucleic acid and/or a selection marker nucleic acid. This may be performed by RT-PCR (including quantitative RT-PCR), Northern hybridisation, and/or microarray analysis. Southern hybridisation and/or PCR may be employed to detect CLE-encoding nucleic acids inserted in the genetically-modified plant genome using primers, such as described herein in the Examples.

For examples of RNA isolation and Northern hybridisation methods, the skilled person is referred to Chapter 3 of PLANT MOLECULAR BIOLOGY: A Laboratory Manual, supra. Southern hybridisation is described, for example, in Chapter 1 of PLANT MOLECULAR BIOLOGY: A Laboratory Manual, supra.

In another aspect, the invention provides a method of breeding a leguminous crop plant, said method including the step of crossing parent leguminous crop plants to produce a progeny leguminous crop plant having a desired trait, wherein at least one of the parent leguminous crop plants is selected as having a desired trait associated with CLE protein-regulated nodulation and/or nitrogen fixation.

By “plant breeding” or “conventional plant breeding” is meant the creation of a new plant variety or cultivar by hybridisation of two donor plants, one of which carries a trait of interest, followed by screening and field selection. Such methods are not reliant upon transformation with recombinant DNA in order to express a desired trait. However, it will be appreciated that in some embodiments, the donor plant may carry the trait of interest as a result of transformation with recombinant DNA which imparts the trait.

It will be appreciated by a person of skill in the art that a method of plant breeding typically comprises identifying a parent plant which comprises at least one genetic element or component associated with or linked to a desired trait associated with nodulation and/or nitrogen fixation. This may include initially determining the genetic variability in CLE proteins or peptides, or encoding nucleic acids or in a CLE gene promoter (e.g., allelic variation, polymorphisms etc.) associated with or linked to nodulation or nitrogen fixation. Depending on the desired trait, those alleles or polymorphisms would be selected for in the plant breeding method of the invention. This may also be facilitated by identification of genetic markers (e.g., AFLPs, RFLPs, SSRs, etc.) associated with the desired trait that are useful in marker-assisted breeding methods.

For example, a parent plant may comprise a genetic element that encodes a variant CLE peptide or CLE gene promoter. In one embodiment, the variant CLE peptide or CLE gene promoter may provide a desired trait in the form of super- or hyper-nodulation. In another embodiment, the variant CLE peptide or CLE gene promoter may provide a desired trait in the form of suppression of nodulation and/or nitrogen fixation, as hereinbefore described. The advantages of super- or hyper-nodulation and suppression of nodulation/nitrogen fixation have been hereinbefore described in relation to genetically-modified plants.

By way of example only, a plant breeding method may include the following steps:

(a) identifying a first parent plant of a leguminous crop plant species and a second parent plant of the same or a different leguminous crop plant species, wherein at least one of the first and second parent plants comprise at least one genetic element associated with or linked to a desired trait associated with CLE protein regulated nodulation and/or nitrogen fixation, and wherein the first and second plants are capable of cross-pollination;

(b) pollinating the first parent plant with pollen from the second parent plant, or pollinating the second parent plant with pollen from the first parent plant;

(c) culturing the leguminous crop plant pollinated in step (b) under conditions to produce progeny leguminous crop plants;

(d) selecting progeny leguminous crop plants that possess the desired trait.

It will be appreciated by those skilled in the art that once progeny plants have been obtained (e.g., F1 hybrids), which may be heterozygous or homozygous, these heterozygous or homozygous plants may be used in further plant breeding (e.g. backcrossing with plants of parental type or further inbreeding of F1 hybrids).

The breeding method of the invention may be applicable to leguminous crop plants such as soybean species of the Glycine genus such as Glycine max and Glycine soja, alfalfa, clovers, mungbean, Phaseolus beans, azukibeans, acacia, Pongamia, Faba beans, lentils, peas, cowpea, pigeonpea, chickpea, lupins, mesquite, carob and peanuts, although without limitation thereto.

In order that the invention may be readily understood and put into practical effect, particular preferred embodiments will now be described by way of the following non-limiting examples.

### EXAMPLES

### Example 1

### Introduction

The structural similarity of GmNARK to CLAVATA1 in Arabidopsis led us to propose that the ligand of GmNARK may be a CLE peptide, similar to the CLV3 ligand that binds to CLAVATA1 (Clark et al., 1997; Fletcher et al., 1999; Ogawa et al., 2008). CLE peptides are required for several plant developmental and regulatory processes, including for the regulation of the shoot (Fletcher et al., 1999) and root (Fiers et al., 2004) apical meristem and vasculature differentiation (Ito et al., 2006). CLE peptides are characterised by a conserved N-terminal signal peptide and C-terminal region of approximately 12 amino acids that is proposed to act as the final active product.

Significant modification of CLE peptides is thought to occur post-translationally, including proteolytic cleavage (Ni & Clark, 2006), hydroxylation (Kondo et al., 2006) and glycosylation (Ohyama et al., 2009). There is some debate in the literature but the best evidence to date indicates that CLV3 functions as a 12 or 13 amino acid arabinosylated peptide (Kondo et al., 2006; Ohyama et al., 2009).

To date, CLE peptides have been identified that are induced specifically by rhizobia or by either rhizobia or nitrate. These have all been shown to reduce nodulation in L. japonicus and M. truncatula when ectopically over-expressed in transgenic roots. Whereas the L. japonicus peptides only reduced nodulation effectively in wild-type plants (Okamoto et al., 2009), the M. truncatula peptides exhibited inhibition in hypernodulating Mtsunn plants (Mortier et al., 2010). This indicates that they may not regulate nodulation solely through Mtsunn and the systemic AON mechanism. Alternatively they used a weak allele of Mtsunn. Indeed, the large number of LRR receptor-like proteins and CLE ligands may suggest that certain CLE peptides can bind to more than one LRR receptor to regulate plant development.

We investigated the function of CLE peptides in the regulation of soybean nodulation. Using BLAST searches, we identified three CLE peptide genes that exhibited either rhizobia Nod factor- or nitrate-induced gene expression. We designated these CLE30, CLE60 and CLE80. We show that over-expression of the inoculation dependent CLEs (CLE30, CLE80) completely abolishes soybean nodulation in both a systemic and NARK-dependent manner. In contrast, over-expression of the nitrate-responsive CLE (CLE60) only partially reduces nodulation, and appears to function in a local, yet still NARK-dependent, manner. Grafting experiments using wild type and nark mutant plants grown under several nitrate conditions confirmed this local role for NARK in nitrate-regulation of nodulation. We also show, for the first time, differences in the localisation of the nodule inhibition responses induced by inoculation or nitrate-responsive CLE peptides. We propose that these differences may account for the different yet overlapping roles of nitrate and inoculation dependent regulation of nodulation in soybean and other crop legumes.

### Experimental Procedures

### General Plant and Bacterial Growth Conditions

Soybean (Glycine max) lines used include the wild type Williams and its isogenic supernodulating nark mutant lines, nod4 and nod3-7 and the wild type Bragg and its isogenic hypernodulating nark mutant line, nts1116. Plants were grown in controlled glasshouse conditions (28° C./26° C. day/night, 16 hour day) in autoclaved pots and vermiculite where sterile growing conditions were required. Seeds requiring sterilisation were treated with 70% ethanol, 3% H₂O₂ for 1 min, followed by several rinses with water. Plants were watered as required with a modified nutrient solution lacking nitrogen (Herridge, 1982). Plants were inoculated with approximately OD₆₀₀ 0.01 Bradyrhizobium japonicum CB1809 or the corresponding NF mutant nodU grown in yeast-mannitol broth (YMB) at 28° for two days. For nitrate treatments, plants were watered with the indicated solutions of KNO₃ every two days. For grafting experiments, reciprocal grafting was carried out 8 d after sowing and the graft unions were allowed to recover for 6 d before B. japonicum inoculation. Nodulation was scored three weeks after inoculation.

### Bioinformatic Analysis

Candidate inoculation or nitrate dependent CLE genes were identified by BLASTp searches of the Medicago expression atlas (Benedito et al., 2008) and soybean resources at NCBI and available through the Soybean Genome Project (www.phytozome.net/soybean; Schmutz et al., 2010). The conserved C-terminal CLE motif of LjRS1/2 was used as the initial query sequence. Further searches were undertaken using the initial candidates to identify additional gene candidates and their genomic environment. Where partial sequences were obtained, the complete CDS was predicted from gene models available via Phytozome or as determined by gene prediction programs (Burge & Karlin, 1997; Salamov & Solovyev, 2000). Multiple sequence alignments (Clustal X2, Larkin et al., 2007) and signal peptide predictions (SignalP 3.0, Bendtsen et al., 2004) were carried out using the soybean peptide sequences.

### Molecular Biology

The full length CDS of CLE30, CLE60 and CLE80 was directionally cloned into the pKANNIBAL vector for expression of RNAi constructs (Wesley et al., 2001). Pfu polymerase (Stratagene, La Jolla, Calif.) was used to amplify PCR products incorporating restriction nuclease sites from cDNA samples shown to express the target gene. XhoI/EcoRI (for) and KpnI (rev) restriction sites were included in the primer sequences depending on internal restriction sites of each gene (full primer sequences are included in Table 2). Likely clones were confirmed by direct DNA sequencing and capillary separation. Constructs were sub-cloned into p15SRK2 or pM1KCK1 integration vectors (A. Kereszt, unpublished) as a NotI fragment before tri-parental mating to introduce the constructs into Agrobacterium rhizogenes K599. For transgenic root verification in the CLE60 over-expression experiments the DsRed reporter was subcloned into pM1KCK1 from pHairyRed, which is a modified version of pCAMBIA (M-H Lin, P M Gresshoff, B J Ferguson, unpublished). Tri-parental mating was used to introduce DsRed into A. rhizogenes K599 carrying the CLE60 construct. A. rhizogenes were subsequently used for the induction of transgenic soybean hairy roots according to Kereszt et al (2007).

### RT-qPCR Analysis

For gene expression analyses, tissue was snap frozen and homogenized in liquid nitrogen using a mortar and pestle. RNA was extracted using TRIzol reagent according to the manufacturer's instructions (Invitrogen). DNAse treatment (Fermentas, Ontario) was performed on 1 μg of RNA in the presence of MgCL₂. cDNA was synthesised with SuperScript III (Invitrogen) from 0.5 μg DNAse treated RNA. RT-qPCR was conducted using an ABI 7900HT cycler in 384 well plates with SYBR green fluorescence detection. All reactions were performed in duplicate for at least three biological replicates using intron-spanning primers where possible and a target amplicon of approximately 100 bp (full primer sequences are included in Table 2). Gene expression values were calculated relative to ATP synthase after normalisation for PCR efficiency and plot correlation (R2) values of each primer pair as determined by LinRegPCR 7.5 (Ramakers et al., 2003).

### Results

### Identification of Rhizobia Inoculation and Nitrate Responsive CLE Peptides

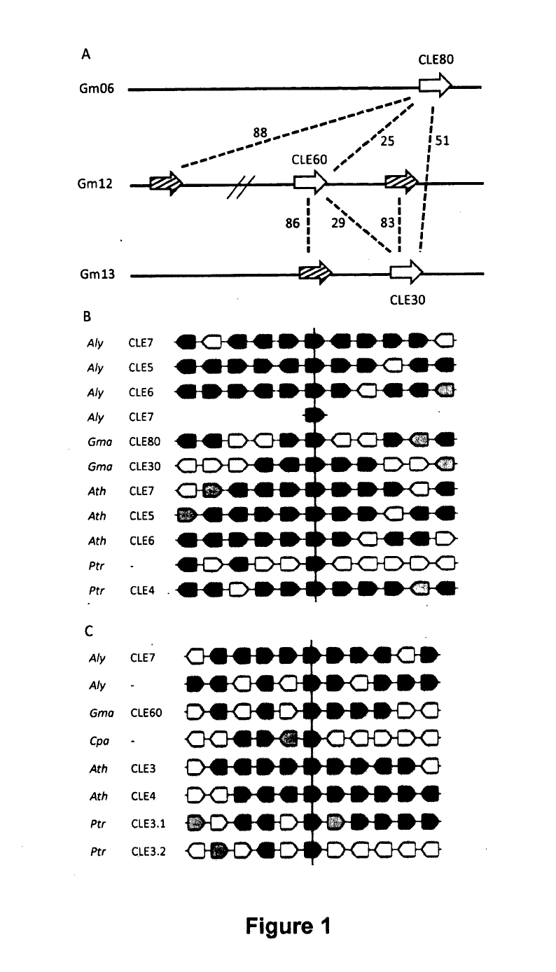

To identify candidates for inoculation or nitrate dependent CLE peptides involved in nodule regulation, searches were undertaken based on sequence homology with the LjRS1/2 protein sequences. BLAST searches identified two M. truncatula genes up-regulated in response to inoculation in the Medicago gene atlas (Benedito et al., 2008) which have subsequently been shown to reduce nodulation when over-expressed (Mortier et al., 2010). The peptide sequence of all rhizobia inoculation-dependent legume CLE peptides was aligned using ClustalX2 to produce a consensus sequence (Larkin et al., 2007). Publicly available soybean genome sequences (Phytozome, Schmutz et al., 2010) and EST sequences (NCBI) were searched to identify soybean sequences sharing homology with this consensus sequence or with the L. japonicus and M. truncatula CLE sequences individually. Searches focused on genes that shared the greatest similarity with the C-terminal CLE domain. Three soybean CLE genes were subsequently identified as candidates for either rhizobia inoculation or nitrate response. These genes were designated CLE30, CLE60 and CLE80 according to the last two digits of their Phytozome gene identifier (FIGS. 1, 2).

Further BLAST searches conducted in soybean identified a highly similar homologue for each of the three CLE genes likely resulting from the ancestral soybean allopolyploidisation and neo-diversification events (FIG. 1). Primers specific to each of the genes has indicated that the duplicate copies in each case are inactive or have greatly reduced expression in RT-qPCR studies (data not shown). CLE30 and CLE60 are located in syntenous regions of chromosome 13 and 12 respectively. In each case the duplicate is located on the syntenous chromosome region adjacent to the actively expressed CLE (FIG. 1). CLE80 has a homologue in a syntenous region of chromosome 12 independent of this locus (FIG. 1).

### Sequence Characteristics of Nodulation CLE Peptides

Peptide elicitors of AON are predicted to require export from the cell to the xylem that is dependent on an N-terminal signal peptide. SignalP 3.0 analysis (Bendtsen et al., 2004) showed CLE30, CLE60 and CLE80 are all predicted to possess an N-terminal signal peptide comprising approximately 30 hydrophobic amino acids, with the most likely cleavage site for the signal peptide being after 28, 29 and 26 amino acids for CLE30, CLE60 and CLE80 respectively (FIG. 2a). This analysis also showed that the signal peptide region of the inoculation-dependent CLE peptides shared areas of high conservation including a motif of ‘STFFMTLQAR’ preceding the predicted signal peptide cleavage site. CLE60 showed the most significant divergence from this motif lacking several nucleophilic and hydrophobic amino acids. Well-conserved proline residues were also identified close to the C-terminus of the inoculation-dependent CLEs (FIG. 2b).

### Expression Pattern of GmCLE Genes

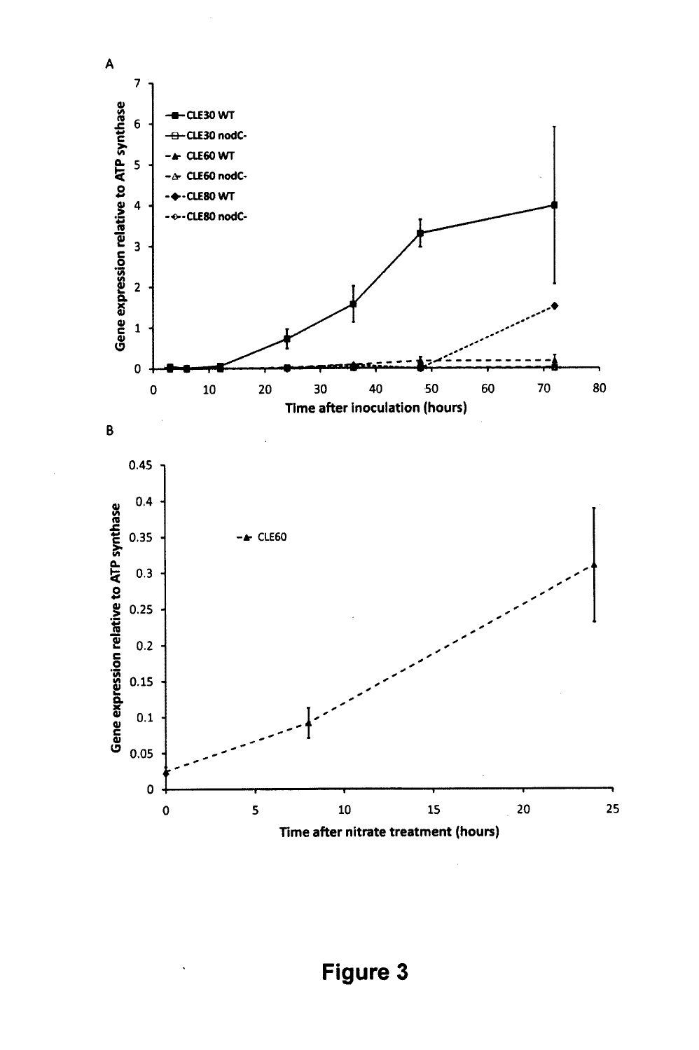

Soybean tap root tissue corresponding to the area of root-hair emergence at the time of rhizobia-inoculation was harvested in a time-course manner to identify genes responding to early infection events. RT-qPCR studies using this tissue showed that CLE30 expression is significantly up-regulated in response to inoculation with compatible wild-type rhizobia relative to incompatible nodC (chitin synthase) mutant rhizobia (FIG. 3a), which are unable to produce Nod factor. This difference in expression is significant as early as 12 h after inoculation and increased until the last tissue collection time in this study at 72 h. CLE60 expression also responded to inoculation, however this was not detectable until 48 hours after inoculation and was much weaker relative to CLE30 expression (FIG. 3a). CLE80 expression was not significantly changed until 72 hours after inoculation but was induced strongly at this point (FIG. 3a).

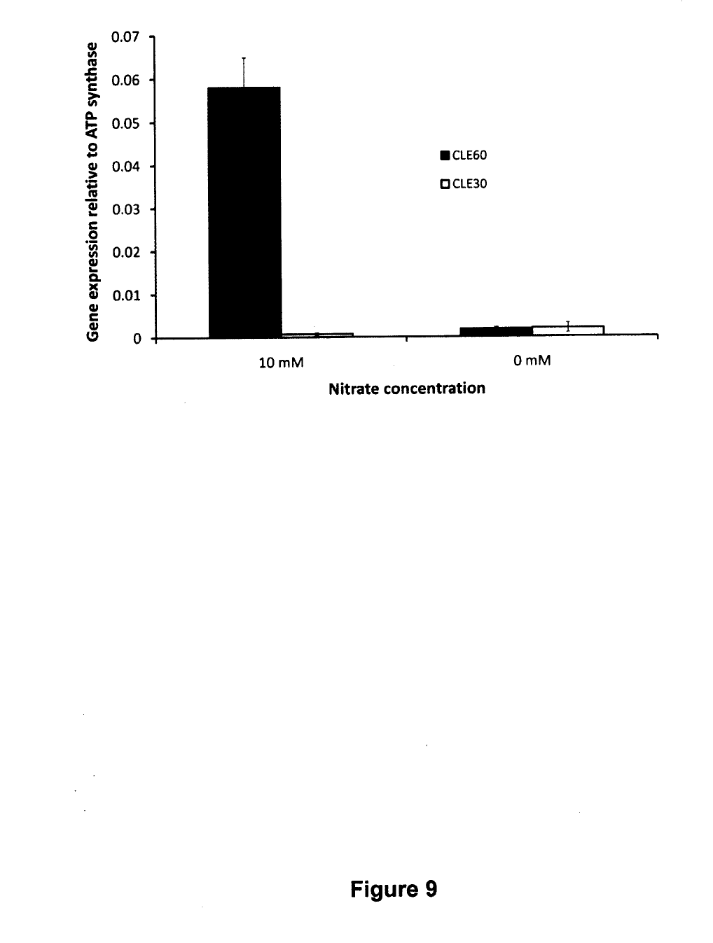

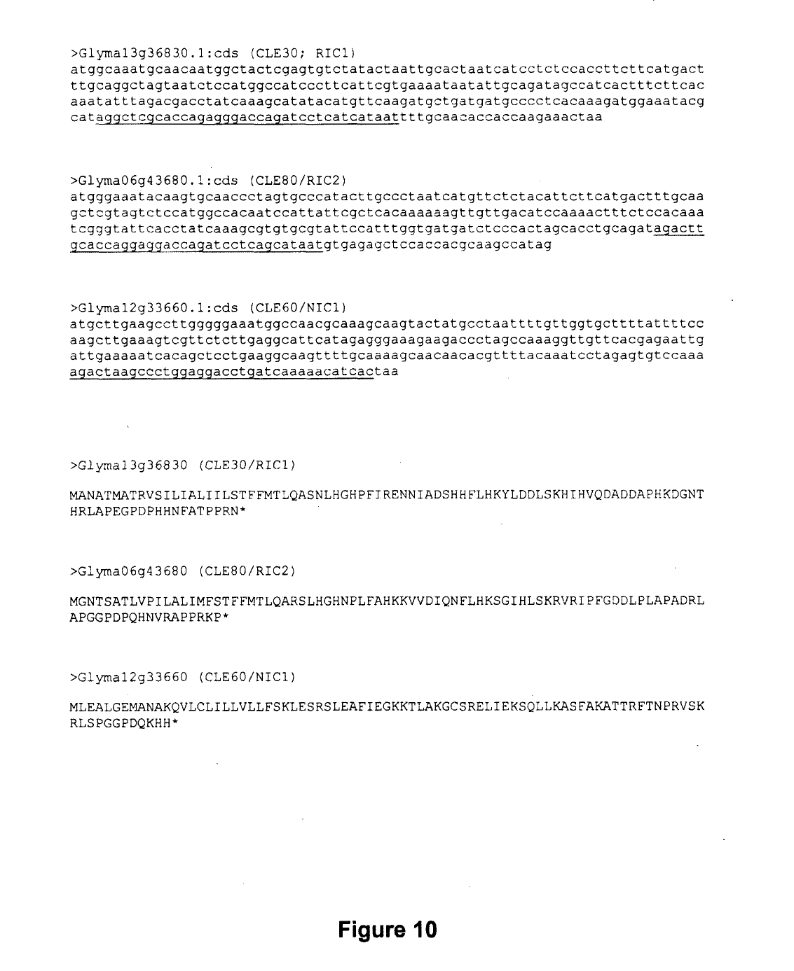

In a preliminary study to determine if nitrate can induce the expression of the soybean CLE genes, root tissue was harvested after treatment with either 0 mM (water control) or 10 mM KNO₃ for two weeks. Using RT-qPCR, CLE60 expression was significantly induced by nitrate relative to the control, whereas CLE30 expression was unchanged (FIG. 9). A more thorough study to identify the induction of CLE60 expression by nitrate was subsequently performed using, root tissue harvested at 0, 8 or 24 h after the commencement of the above treatments. After 8 h of nitrate treatment, CLE60 expression was significantly elevated and increased further by 24 h (FIG. 3b).

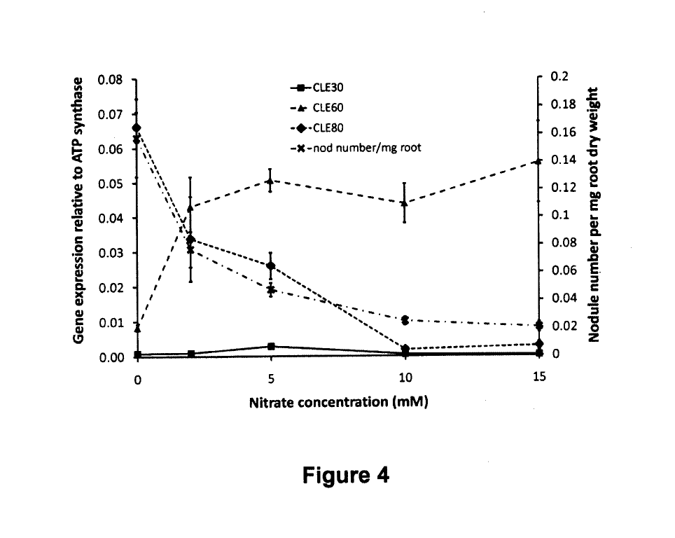

To determine if CLE60 expression correlated with the inhibition of nodulation cause by nitrate, various nitrate concentrations were applied. Soybean plants were treated with either 0, 2, 5, 10 or 15 mM KNO₃ for 2 days prior to, and for 2 weeks following, rhizobia inoculation. The treated plants were then harvested simultaneously for RT-qPCR and nodule count studies. This experiment demonstrated that CLE60 was significantly induced by KNO₃ at levels as low as 2 mM and reached a plateau after 5 mM treatment, while nodule numbers were reduced in an inverse manner (FIG. 4). In contrast to the early inoculation time course data, CLE30 expression did not appear to correlate with mature nodule numbers, whereas CLE80 expression correlated strongly, exhibiting reduced expression levels at higher nitrate concentrations (FIG. 4).

Similar to nodulation, the systemic regulation of root arbuscular mycorrhizal infection acts through GmNARK in the shoot (Meixner et al., 2005). To determine if any of the three soybean CLEs were upregulated by arbuscular mycorrhizal infection and thus had a role in their regulation, RT-qPCR was performed using cDNA from Glomus intraradices infected soybean roots. No significant change in the expression of the CLE genes relative to uninfected plants was observed at 5, 14 or 70 d after infection with Glomus intraradices in either wild type Bragg or nts1116 supernodulating mutant plants.

### Cloning and Transgenic Expression of GmCLE Genes

To determine if the soybean CLE peptides regulate nodulation in a NARK-dependent manner, a reverse-genetics approach using a hairy-root transformation system was used. Over-expression of a systemic Q signal would be expected to inhibit nodulation, even in non-transformed roots. To test this, the complete CDS of CLE30, CLE60 or CLE80 was cloned into the pKANNIBAL vector for the expression of efficient RNAi constructs (Wesley et al., 2001). However, to generate over-expressing constructs, the antisense construct was omitted and the Pdk intron of pKANNIBAL was left intact. Previous studies have shown the inclusion of an intron can lead to higher and more consistent expression levels in transgenic plants (Norris et al., 1993).

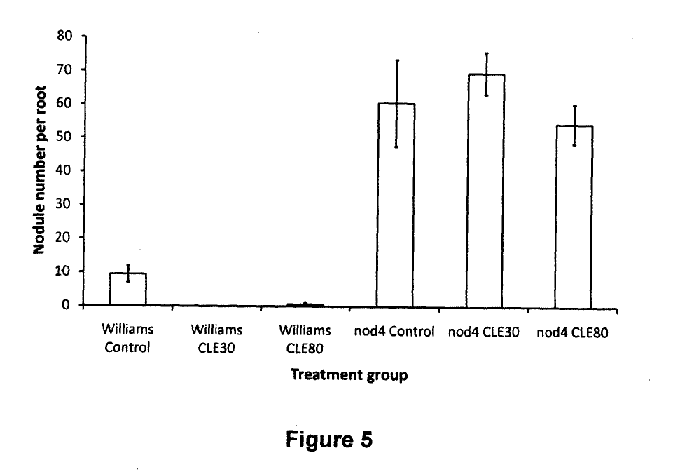

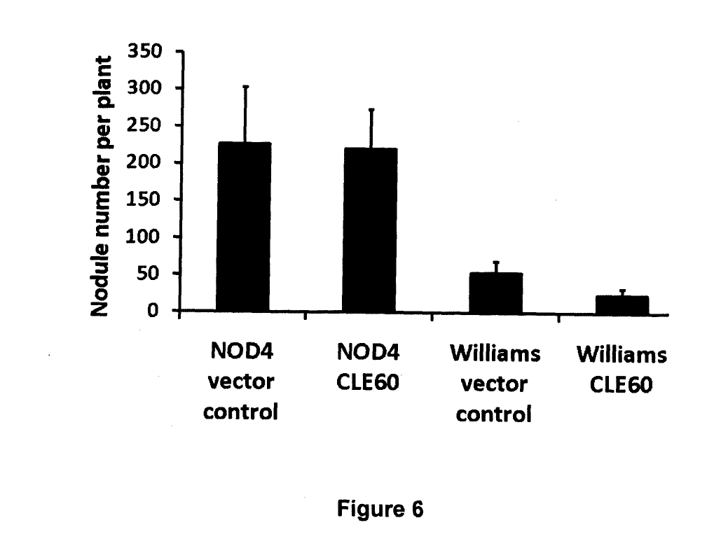

Tri-parental mating was used to integrate the constructs into Agrobacterium rhizogenes K599 which induces the formation of transgenic hairy roots in soybean (Kereszt et al., 2007). The use of integrative vectors has been shown to increase the transformation efficiency relative to binary vectors commonly used in plant transformation. Over-expression of either CLE80 or CLE30 completely eliminated nodulation in wild-type soybean plants. In contrast, there was no reduction in the nodule numbers of equivalent nod4 supernodulation nark mutant plants relative to vector-only control plants (FIG. 5). Over-expression of CLE60 reduced nodulation by approximately half relative to vector-only transformed control plants (FIG. 6). There was no corresponding reduction in nodulation in nod4 plants between the two treatments (FIG. 6).

### Nitrate Regulation of Nodulation

The strong systemic effect of AON has complicated the understanding of nitrate control of nodulation. Several studies have indicated that nitrate regulation is determined by the shoot (Day et al., 1989; Francisco & Akao, 1993), while others have highlighted the role of a local regulation in the root (Carroll & Mathews, 1990). In fact, both acting in parallel have recently been shown to be important for nodulation (Jeudy et al., 2010) and may regulate nodulation differently. We re-examined these data sets that investigated AON-deficient plants and found that there may have been a nitrate-induced effect of the root-genotype on nodulation that was masked by the lack of AON in the shoot. Therefore, to determine if NARK plays a local role in nitrate regulation of nodulation we investigated the nodule numbers of wild-type (Williams) and supernodulation (nod3-7 and nod4) nts mutant plants that were grafted and treated with high or low levels of nitrate. Plants with AON intact (WT/nts and WT/WT) were used to assess if NARK functions in the root as part of a local nitrate-controlled regulation of nodulation.

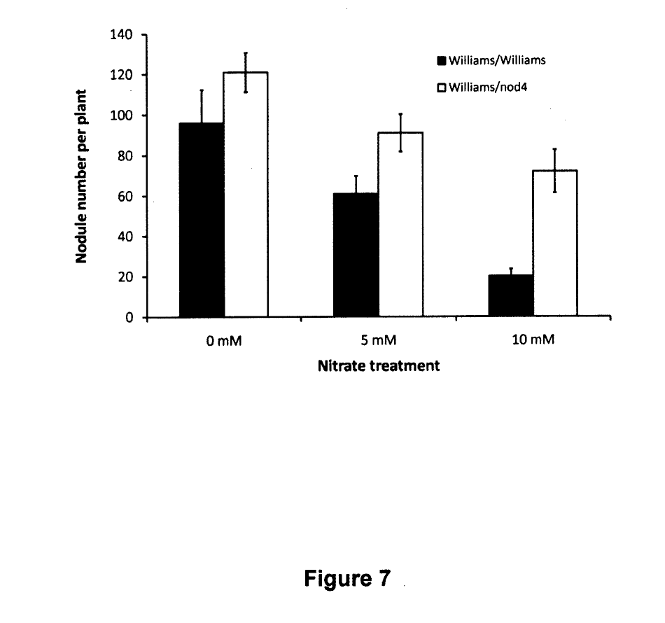

When treated with 5 mM or 10 mM KNO₃ nodule numbers were reduced significantly (P=0.02 and P=0.002 respectively) in grafted plants having a wild-type rootstock compared to those having a nod4 mutant rootstock (FIG. 7). There was no significant difference in nodule numbers when no nitrate was applied (P=0.23). In concordance with previous studies (Delves et al., 1986; Delves et al., 1987), there was no difference observed in nodule numbers between the AON defective grafting combinations (nts/WT and nts/nts) at any nitrate concentration (data not shown).

### Discussion

Based on homology to CLE peptides that are necessary for nodule regulation in other legumes, we identified three nitrate or inoculation responsive CLEs in soybean. These three CLEs all regulate soybean nodulation in a NARK-dependent manner, although there are differences in their expression response and in the localisation of their action (Table 1). The soybean CLEs share a high level of conservation in the CLE domain and have well-conserved motifs within the signal peptide and at the C-terminal.

Gene expression analyses showed that CLE30 and CLE80 are induced in response to Nod factor produced by compatible Rhizobium inoculation, however, differences in the timing of their induced expression suggest that they are not wholly redundant. CLE30 is expressed early, possibly in response to the initial signalling and cell division events induced following Bradyrhizobium inoculation. This expression pattern is consistent with that of LjRS1/2 and MtCLE13 which are reported to respond quickly to inoculation (Mortier et al., 2010; Okamoto et al., 2009). In the case of MtCLE13, expression was also observed in the ERN/mutant bit1-1 (Mortier et al., 2010), which is defective in the later stages of Nod factor signalling (Andriankaja et al., 2007; Middleton et al., 2007). In contrast, CLE80 expression correlates with the emergence of more mature nodule structures, similar to the expression of MtCLE12 (Mortier et al., 2010). It may therefore be induced following the onset of the nodule meristem or in response to nitrogen fixation. That multiple signals may act in parallel to regulate nodule numbers is not surprising in light of the findings by Li et al. (2009) that AON in pea may be activated at several developmental stages of nodulation.

Over-expression of both CLE30 and CLE80 was sufficient to elicit a systemic AON response and in both cases eliminated nodulation completely in wild-type plants. The complete lack of nodule inhibition in Gmnark mutant plants suggests that these CLEs are functioning in AON through NARK, possibly as the Q signal described in various AON models (Ferguson et al., 2010; Magori & Kawaguchi, 2009). Future experiments aimed at directly detecting the transport and function of these CLE peptides will help to verify these models. That the legume CLEs have different expression patterns and timing appears to indicate that NARK and its orthologues have evolved as a common receptor in various nodule regulation mechanisms. This would explain the dual role of NARK in AON and nitrate regulation of nodulation (Carroll et al., 1985a; b; Oka-Kira & Kawaguchi, 2006). That none of the CLE genes were regulated by AMF infection, despite this symbiosis also being regulated through NARK (Meixner et al., 2005; Meixner et al., 2007), may indicate that other related CLE peptides are involved in systemic mycorrhizal control.

LjRS2 was reported to be responsive to both nitrate treatment and rhizobia inoculation and it exhibited a systemic regulation response when over-expressed in L. japonicus hairy-roots (Okamoto et al., 2009). This led to the proposal that LjRS2 could systemically induce nodule regulation in response to both inoculation and nitrate in L. japonicus. In contrast, we found that CLE60 was induced in response to nitrate but was not substantially altered by effective Bradyrhizobium inoculation. Moreover, it appears to function locally in the root and not systemically. Significant CLE60 expression was detected at the lowest nitrate levels we tested (2 mM KNO₃) and reached a plateau at 5 mM KNO₃. Although there was a small increase in CLE60 expression 48 h after Bradyrhizobium inoculation, the increase was minimal compared with that detected following nitrate treatment. It was also dramatically lower than that detected for CLE30 following Bradyrhizobium inoculation. It is possible that the small change is a promoter artefact rather than a true response and CLE60 is not likely to be involved in inoculation-dependent nodule regulation. Over-expression of CLE60 caused an approximate 50% reduction in nodulation, indicating that it is highly capable of nodule inhibition, although not to the same degree as the inoculation induced CLEs. However, what is consistent with the Bradyrhizobium-induced CLE peptides is that CLE60-inhibition of nodulation was NARK-dependent. Taken together with the grafting experiments highlighting the local role for NARK in nitrate regulation of nodulation this indicates that CLE60 is likely a local inducer of NARK-dependent nodule regulation independent of the systemic AON mechanism. CLE60 is thus the first CLE peptide shown to be capable of nodule regulation via NARK that is independent of rhizobia inoculation.

Increasingly it appears that motifs outside of the CLE domain are required for the in planta function and specificity of CLE peptides. CLE activity is likely related to tissue specificity of the promoters, tissue localisation caused by signal peptide trafficking and post-translational modifications of the final peptide. The receptor specificity is likely due to the distinct structure of each CLE peptide (Meng et at, 2010) where most CLE peptides appear to share at least basic structural similarities due to a bend imparted by central glycine and proline residues (Cock & McCormick, 2001). The putative Q CLEs have distinct double central G residues flanked by proline which may impart AON receptor specificity. In Arabidopsis, CLE function is dependent on motifs outside of the CLE domain, particularly in the signal peptide (Meng et al., 2010). This is consistent with findings regarding the activity of CLEs produced by soybean cyst nematodes (Wang et al., 2010). Our work in soybean indicates that the signal peptide may be critical in differentiating the tissue specificity of the nitrate-responsive CLE from the inoculation-responsive CLEs. Aligning the sequences of the Bradyrhizobium-induced CLEs to the previously reported inoculation-dependent CLE sequences allowed us to extend the conserved motif (STFFMTLQAR) within the signal peptide that was first identified by Okamoto et al. (2009) and has not previously been reported outside of legumes. We found this motif was common amongst all the legume CLE peptides studied, with the exception of MtCLE12, where it was partially conserved, and GmCLE60, where it was not well conserved. Interestingly, the inoculation-dependent soybean CLEs and MtCLE13 also possessed the conserved proline residues close to the C-termini observed in LjRS1/2 (Okamoto et al., 2009).

The ability of the soybean CLE peptides to inhibit nodulation in wild type, but not in nark mutants, suggests that they are functioning via NARK to regulate nodulation. It appears that between the CLEs identified in each of the three legume species subtle differences exist in the manner in which inoculation and nitrate induced CLEs function and alternate receptors may exist to modulate their signalling. In particular, the CLE peptides identified in M. truncatula maintain some inhibitory ability in Mtsunn plants and may be required for nodule development signalling through alternative LRR-RLKs (Mortier et al., 2010).

Previous studies have shown that nitrate treatment of one side of a split root system does not systemically change the nodule mass on the other side of the split root system (Tanaka et al., 1985). However, in the same experiment nitrogen fixation was found to be systemically reduced. This regulation may be due to amino acid transport in the phloem (Sulieman et al., 2010). We have extended this understanding of a local role of nitrate regulation in legume nodulation by demonstrating a local requirement for NARK in nodulation regulation that is dependent on CLE60. This appears to account for why NARK is expressed in the root, despite it previously being believed to only be effective in the shoot (Nontachaiyapoom et al., 2007). Our grafting experiments also demonstrate that nitrate regulation of nodulation acts locally through NARK in the root, as the presence of NARK in the shoot alone is not sufficient to regulate nodulation following nitrate treatment. This may vary between legume species as it has been reported that the nitrate responsive CLE peptide, LjRS2, can inhibit nodulation systemically in L. japonicus. The absence of several residues in the CLE60 signal peptide that are conserved in the inoculation-dependent CLEs may be responsible for its lack of systemic response.

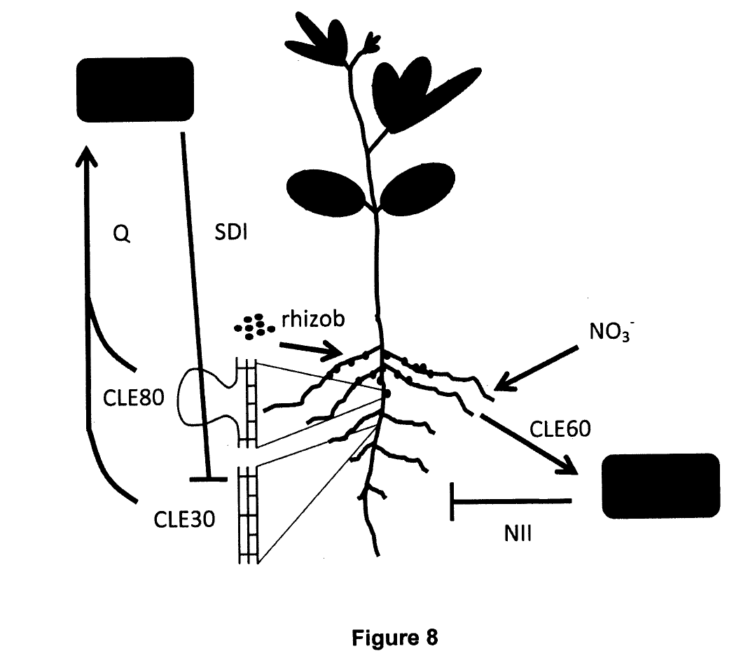

We propose an enhanced AON model in soybean where there is both a systemic and local role for NARK involving multiple CLE peptide ligands (FIG. 8). Future experiments will focus on detecting the peptides in planta to identify both their active and transport forms, in addition to studies, such as site-directed mutagenesis of critical amino acids within the CLE peptides and outside (i.e., presumed protein clevage sites putatively involved in long distance transport and CLE peptide maturation) aimed at better understanding the role of NARK and CLE60 in the nitrate-dependent regulation of nodulation.

### Example 2

### Introduction

Rhizobia induced CLEs (RIC): RIC1 also referred to herein as CLE30; and RIC2 also referred to herein as CLE60; have been shown to function systemically and are produced in response to rhizobia induced nodulation events.

The aim of the work described in this Example was to establish whether GmRIC1 (CLE30) can function to inhibit nodulation in other legume species. This was done by overexpressing GmRIC1 (CLE30 nucleic acid) in the roots of ancestral soybean (Glycine soja) and common bean (Phaseolus vulgaris). Soybean was used as a control as RIC1 (CLE30) has been cloned from soybean and is already known to have an effect in soybean. For common bean, the predicted NARK mutant was used to show that overexpressing RIC1 (CLE30) would not affect nodule numbers in the mutant as it is unable to perceive the signal molecule.

Transformation was done using the hairy root method. Agrobacterium rhizogenes containing a vector with the RIC1 gene (CLE30) and 35s promoter was injected into the stems of young plants. Once transgenic ‘hairy roots’ began growing from the stems the primary roots were removed and the plants with the transgenic roots were replanted and inoculated with the appropriate rhizobia strain.

### Methods

### Overexpression of GmRIC1

### Preparation of Binary Vector

The pKannibal vector containing GmRIC1 was extracted from an overnight liquid culture of E. coli DH5α using a miniprep kit. The pKannibal vector was digested with restriction enzymes NotI and EcoRV to produce two vector fragments and the GmRIC1 insert. The product was run on a 1% agarose gel (6 g agarose powder, 60 mL TAE buffer, 2 mL Ethidium Bromide) and the band the size of the RIC1 insert (3290 bp) was excised and purified using a Promega plasmid extraction kit. The binary vector pART27 was extracted from an overnight liquid culture of DH5α using a miniprep kit. The GmRIC1 fragment was ligated into the NotI sites of the binary vector pART27. Electrocompetent E. coli was transformed with the pART27::GmRIC1 vector using electroporation. E. coli containing the construct of interest were selected from media containing Kanamycin.

### Preparation of Electrocompetent Agrobacterium

To prepare electrocompetent cells, 250 mL of Agrobacterium culture (OD₆₀₀=0.5-0.9) was chilled in water-ice for 10 mins. The cold culture was poured into cold centrifuge tubes and centrifuged at 4° C. and 4,000 rpm for 20 mins, the supernatant was discarded and the cells were resuspended in 200 mL cold, sterile MilliQ (MQ) H₂O. This was repeated twice, after the final rinse the supernatant was discarded and the cells were resuspended in 2 mL cold, sterile 10% glycerol rather than MQ H₂O. The suspension was aliquoted, 60 μL per Eppendorf tube, frozen in liquid nitrogen and stored at −80° C.

### Plant Material

All plants were grown in and in 30 well seed trays of grade 3 vermiculite. G. soja seeds were scarified and soaked in MQ H₂O before planting. All plants were grown in growth cabinets under 16 h day and 8 h night conditions. P. vulgaris and G. max were grown at 28° C. G. soja were grown at 24° C. All plants were fertilised with a no nitrogen fertiliser and B&D solution (Broughton & Dilworth 1971). Table 3 shows a detailed summary of the different species, their mutants and other specifics for each species.

### Hairy Root Transformation

A. rhizogenes K599 with either p15SRK2::GmRIC1 ox or p15SRK2 were grown on LB media (10 g/L Tryptone, 5 g/L Yeast extract, 10 g/L NaCl) for 2 days at 28° C. The bacteria were harvested by scraping it from the agar plate. The harvested bacteria were suspended in 3 mL MQ H₂O, ensuring no lumps remained.

When all plants had unfolded cotyledons they were injected at the hypocotyl as close to the junction of the cotyledons as possible with the A. rhizogenes solutions (Kereszt et al, 2007; Estrada-Navarrete, 2006). Half of each of the WT and mutant lines received A. rhizogenes p15SRK2+GmRIC1 OX and the other half of the plants received A. rhizogenes p15SRK2; Totalling 4 treatment groups.

The infection technique was the same for all species using A. rhizogenes K599. To increase the likelihood of the K599 producing hairy roots, the A. rhizogenes was inoculated with 1 mL 200 μM acetosyringone one hour prior to harvesting the bacteria. After infection all seed trays were misted with H₂O, covered and sealed to ensure high humidity.

Once root tips were visible at the point of infection, the primary roots of the plants were removed and the hairy roots were re-planted. Plants were left in the growth cabinets for one week to form an adequate root system. The plants were subsequently transferred to the glasshouse and re-potted into larger pots. After re-potting plants were also inoculated with the appropriate strain of rhizobia for that particular species. Plants were watered and fertilised with B&D solution (Broughton & Dilworth 1971). After 2-3 weeks plants were uprooted and the nodules counted.

### Results

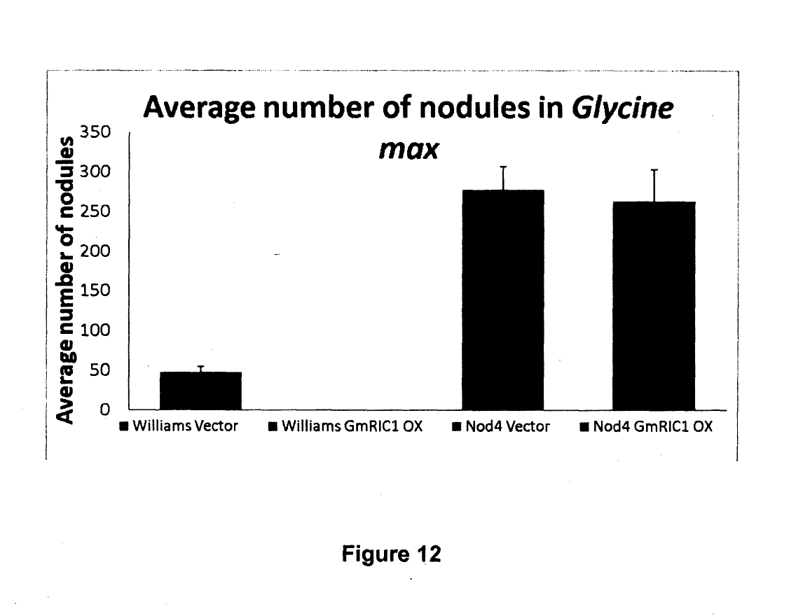

G. max was used as a control species to represent what the effect of GmRIC1 should look like if it has an effect in other species. The results show (FIG. 12) that in the WT, GmRIC1 completely inhibits nodulation and in the WT empty vector treatment the plants form normal numbers of nodules. When GmRIC1 is overexpressed in G. max nod4 plants lacking functional NARK, nodule numbers were unchanged between the treatment groups, with both displaying supernodulating phenotypes.

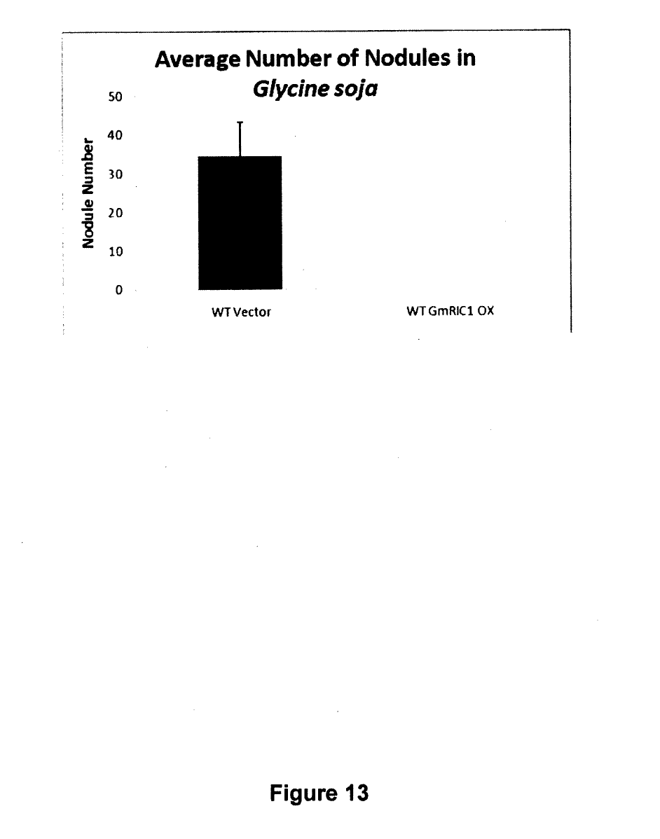

Overexpression of GmRIC1 in G. soja (FIG. 13) completely inhibited nodulation. In contrast, WT G. soja plants transformed with the vector only displayed normal nodulation (FIG. 13).

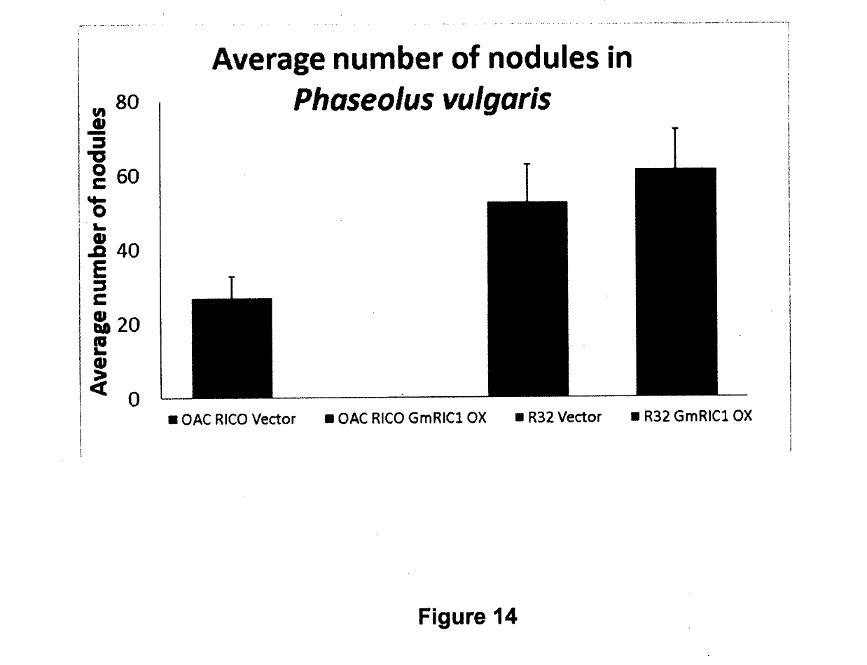

Overexpression of GmRIC1 in P. vulgaris WT cv. OAC RICO resulted in inhibition of nodulation compared with WT plants transformed with the vector only (FIG. 14). In contrast, there was no significant effect of GmRIC1 overexpression on nodulation in the R32 supemodulation mutant compared with R32 plants transformed with the vector only (FIG. 14).

### Discussion

GmNARK and its orthologues MtSUNN (Schnabel et al 2005), LjHAR1 (Nishimura 2002) and PsSYM29 (Sagan and Duc 1996, Krusell et al. 2002) share very high levels of homology. In a study by Lin et al (2010) leaf extract was made from S. Meliloti inoculated M. truncatula WT and AON mutant plants. Leaf extract from the WT plants was found to suppress hypernodulation in G. max, whereas leaf extract harvested from the AON mutants had no effect. This study shows that the SDI component of AON is also conserved amongst legumes. The results from this study also strongly suggest that GmRIC1 is able to function in a range of legumes (FIG. 13, FIG. 14). This further suggests that CLE peptides are highly conserved amongst different species of legumes (Okamoto et al., 2009; Mortier et at, 2010) and that the whole AON process must be of high evolutionary importance as it is so highly conserved. The results show that there is complete inhibition of nodulation in G. soja (FIG. 13).

Implications/Relevance of this Study

Nitrogen is one of the most limiting nutrients when it comes to plant growth. Capturing nitrogen from the atmosphere and breaking the triple bond in order to convert it into a form of nitrogen that is able to be used by plants is a very energy expensive process. Energy consumption is at the forefront of most political and environmental debates at the moment as fossil fuel stores begin to run low and by products of energy are continually blamed for the rapidly declining state of the environment. Nodulation and related nitrogen fixation convert atmospheric nitrogen into forms of nitrogen that are available to plant. Research into understanding the processes that control nodulation is a highly important field and can lead to genetically manipulating plants and/or generating highly efficient nodulating plants having applications in agriculture and the biofuel industry.

### Example 3

Nitrate induced CLE (NIC1), also referred to herein as CLE60, functions locally in the roots and is produced in response to nitrate in the soil. Further studies were undertaken to determine whether nitrogen sources other than nitrate could also induce expression of CLE60 (NIC1).

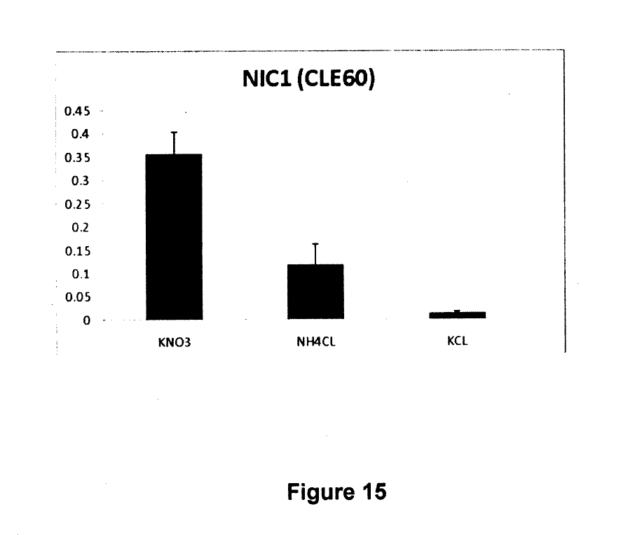

As expected from previous results, treatment with KNO₃ significantly induced expression of CLE60 (GmNIC1) in the roots but not that of CLE80 or CLE30 relative to the KCL control. NH₄Cl also induced expression of CLE60 relative to the control and did not affect expression of CLE30 and CLE80. As can be seen in FIG. 15, the expression of CLE60 in roots treated with ammonium was not as high as those treated with nitrate, however the enhanced expression was statistically significant in both treatments.

### Example 4

As hereinbefore described, the amino acid sequences of SEQ ID NOS:1-3 and consensus sequence SEQ ID NO:4 appear to constitute a “CLE domain” of the larger, fill length CLE protein (such as set forth in SEQ ID NOS:11-13. We therefore undertook experiments to determine which of the amino acids of the CLE domain are required for CLE peptide function.

Site-directed mutagenesis was used to covert each amino acid in the GmRIC1 (CLE30) CLE domain (SEQ ID NO:1) into an alanine (the third amino acid was not altered because it is already an alanine). These constructs were then over-expressed in soybean hairy-roots (using transformation methodology similar to that hereinbefore described) to identify which modifications resulted in reduced CLE activity (i.e. reduced suppression of nodulation).

The underlined amino acids of SEQ ID NO:1 are the amino acids that when replaced with alanine, resulted in reduced suppression: RLAPEGPDPHHNF (SEQ ID NO:1). However, it should also be noted that that amino acids other than those identified based on alanine (A) substitutions are also likely critical. For example, substituting an arginine (R) for the A, or an R for the glycine (G).

In light of these experiments, it is proposed that the corresponding residues may be non-conservatively substituted (e.g. by alanine) in a CLE peptide or CLE domain of a full length protein to thereby produce an interfering mutant protein. Upon introduction of an encoding nucleic acid into a genetically-modified leguminous plant, once expressed this interfering mutant protein may reduce CLE-mediated suppression of nitrogen fixation, thereby acting to enhance or improve nitrogen fixation by the genetically-modified plant.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All patent and scientific literature, computer programs and algorithms referred to in this specification are incorporated herein by reference in their entirety.

TABLE 1
Comparison of legume CLEs capable of nodule regulation

| | | | | Medicago | | | |
|---|---|---|---|---|---|---|---|
| | Glycine max | | | truncatula | | Lotus japonicus | |
| Characteristic | CLE30 | CLE60 | CLE80 | CLE12 | CLE13 | CLE RS1 | CLE RS2 |
| Nitrate induced | no | yes | no | no | no | inhibited | yes |
| Early inoculation | yes | no | no | no | yes | yes | yes |
| Late inoculation | no | no | yes | yes | no | na | na |
| NARK/SUNN/HAR1 | yes | yes | yes | partial | partial | yes | yes |
| dependent inhibition | | | | | | | |
| Systemic activity | yes | no | yes | yes | yes | yes | yes |
| CLE domain 3′ | yes | no | yes | no | yes | yes | yes |
| extension | | | | | | | |
| Signal peptide | yes | no | yes | partial | yes | yes | yes |
| consensus sequence | | | | | | | |

na, Not analysed

TABLE 2
Oligonucleotides used in Example 1.

| Primer | | |
|---|---|---|
| name | Foward primer | Reverse primer |
| CLE30 | GCGCGAATTCCCGC | GCGCGGTACCCGAAATCTA |
| cloning | ATGGCAAATGCAA | CTCCAAACAAAGCTACTT |
| | (SEQ ID NO: 17) | (SEQ ID NO: 18) |
| CLE80 | GCGCCTCGAGATGGGA | GCGCGGTACCACTAT |
| cloning | AATACAAGTGCAACCC | GGCTTGCGTGGTGG |
| | (SEQ ID NO: 19) | (SEQ ID NO: 20) |
| CLE60 | GCGCCTCGAGATGC | GCGCGGTACCTTAGTGA |
| cloning | TTGAAGCCTTGGGG | TGTTTTTGATCAGGTCC |
| | (SEQ ID NO: 21) | (SEQ ID NO: 22) |
| CLE30 | CAAATGCAACA | GCCATGGAGAT |
| RT- | ATGGCTACTCG | TACTAGCCTGC |
| qPCR | (SEQ ID NO: 23) | (SEQ ID NO: 24) |
| CLE80 | GGCCACAATCC | ACGCACACGCT |
| RT- | ATTATTCGCT | TTGATAGGTG |
| qPCR | (SEQ ID NO: 25) | (SEQ ID NO: 26) |
| CLE60 | GCCAAAGGTTG | GCAAAACTTGC |
| RT- | TTCACGAGAA | CTTCAGGAGC |
| qPCR | (SEQ ID NO: 27) | (SEQ ID NO: 28) |

TABLE 3
Summary of methods and plant lines used for each plant species.

| | | | | A. rhizogenes | A. rhizogenes | Removal of | Rhizobia |
|---|---|---|---|---|---|---|---|
| Species | Wild Type | Mutant | Growth Conditions | strain | infection | primary roots | Strain |
| Glycine max | Williams | nod4 | 28° C. | K599 | When | ~7 d post infection | B. japonicum |
| | | | | | cotyledons | | CB1809 |
| | | | | | appear | | |
| Glycine soja | CPI100070 | N/A | 22° C. to 24° C. | K599 | | ~7 d post infection | B. japonicum |
| | | | | | | | CB1809 |
| Phaseolus | OAC RICO | R32 | 28° C. | K599 | | ~15 d post infection | R. phaseoli |
| vulgaris | | | | | | | CC511 |

### REFERENCES

Andriankaja, A., Boisson-Dernier, A., Frances, L., Sauviac, L., Jauneau, A.,
Barker, D. & de Carvalho-Niebel, F. (2007) AP2-ERF transcription factors mediate Nod factor dependent Mt ENOD11 activation in root hairs via a novel cis-regulatory motif. The Plant Cell 19, 2866.
Bendtsen, J. D., Nielsen, H., von Heijne, G. & Brunak, S. (2004) Improved prediction of signal peptides: SignalP 3.0. J. Mol. Biol. 340, 783-795.
Benedito, V. A., Torres-Jerez, I., Murray, J. D., Andriankaja, A., Allen, S., Kakar, K., Wandrey, M., Verdier, J., Zuber, H., Ott, T., Moreau, S., Niebel, A., Frickey, T., Weiller, G., He, J., Dai, X., Zhao, P. X., Tang, Y. & Udvardi, M. K. (2008) A gene expression atlas of the model legume Medicago truncatula. Plant J. 55, 504-513.
Bhuvaneswari, T. V., Bhagwat, A. A. & Bauer, W. D. (1981) Transient susceptibility of root cells in four common legumes to nodulation by Rhizobia. Plant Physiol. 68, 1144-1149.
Bhuvaneswari, T. V., Turgeon, B. G. & Bauer, W. D. (1980) Early events in the infection of soybean (Glycine max L. Merr) by Rhizobium japonicum. Plant Physiol. 66, 1027-1031.
Broughton W J, Dilworth M J (1971). Control of leghaemoglobin synthesis in snake beans. Biochem J 125: 1075-1080.
Burge, C. & Karlin, S. (1997) Prediction of complete gene structures in human genomic DNA. J. Mol. Biol. 268, 78-94.
Caetano-Anollés, G. & Gresshoff, P. M. (1990) Early induction of feedback regulatory responses governing nodulation in soybean. Plant Sci. 71, 69-81.
Calvert, H., Pence, M., Pierce, M., Malik, N. & Bauer, W. (1984) Anatomical analysis of the development and distribution of Rhizobium infections in soybean roots. Can. J. Bot. 62, 2375-2384.
Carroll, B. J. & Mathews, A. (1990) Nitrate inhibition of nodulation in legumes. In Molecular Biology of Symbiotic Nitrogen Fixation., pp. 159-180. Edited by P. M. Gresshof. Boca Raton, Fla.: CRC Press, Inc.
Carroll, B. J., McNeil, D. L. & Gresshoff, P. M. (1985a) Isolation and properties of soybean [Glycine max (L) MERR] mutants that nodulate in the presence of high nitrate concentrations. Proc. Natl. Acad. Sci. U.S.A. 82, 4162-4166.
Carroll, B. J., McNeil, D. L. & Gresshoff, P. M. (1985b) A Supernodulation and nitrate-tolerant symbiotic (nts) soybean mutant. Plant Physiol. 78, 34-40.
Clark, S. E., Williams, R. W. & Meyerowitz, E. M. (1997) The CLAVATA1 gene encodes a putative receptor kinase that controls shoot and floral meristem size in Arabidopsis. Cell 89, 575-585.
Cock, J. & McCormick, S. (2001) A large family of genes that share homology with CLAVATA3. Plant Physiol. 126, 939-942.
Crooks, G., Hon, G., Chandonia, J. & Brenner, S. (2004) WebLogo: a sequence logo generator. Genome Res. 14, 1188-1190.
Day, D. A., Carroll, B. J., Delves, A. C. & Gresshoff, P. M. (1989) Relationship between autoregulation and nitrate inhibition of nodulation in soybeans. Physiol. Plant 75, 37-42.
Delves, A. C., Higgins, A. V. & Gresshoff, P. M. (1987) Shoot control of supernodulation in a number of mutant soybeans, Glycine max (L.) Men. Aust. J. Plant Physiol 14, 689-694.
Delves, A. C., Mathews, A., Day, D. A., Carter, A. S., Carroll, B. J. & Gresshoff, P. M. (1986) Regulation of the soybean-Rhizobium nodule symbiosis by shoot and root factors. Plant Physiol. 82, 588-590.
Estrada-Navarrete G., Alvarado-Affantranger X., Olivares J. E., Diaz-Camino C., Santana O., Murillo E., Guilien G., Sanchez-Guevara N., Acosta J., Quinto C., Li D. X., Gresshoff P. M. & Sanchez F. (2006). Agrobacterium rhizogenes transformation of the Phaseolus spp.: A tool for functional genomics. Mol. Plant-Microbe Interact., 19, 1385-1393.
Ferguson, B. J., Indrasumunar, A., Hayashi, S., Lin, M.-H., Lin, V.-H., Reid, D. E. & Gresshoff, P. M. (2010) Molecular analysis of legume nodule development and autoregulation. J. Integr. Plant Biol. 52, 61-76.
Ferguson, B. J. & Mathesius, U. (2003) Signaling interactions during nodule development. J. Plant Growth Regul. 22, 47-72.
Fiers, M., Hause, G., Boutilier, K., Casamitjana-Martinez, E., Weijers, D., Offringa, R., van der Geest, L., van Lookeren Campagne, M. & Liu, C.-M. (2004) Mis-expression of the CLV3/ESR-like gene CLE19 in Arabidopsis leads to a consumption of root meristem. Gene 327, 37-49.
Fletcher, J. C., Brand, U., Running, M. P., Simon, R. & Meyerowitz, E. M. (1999) Signaling of cell fate decisions by CLAVATA3 in Arabidopsis shoot meristems. Science 283, 1911-1914,
Francisco, P. & Akao, S. (1993) Autoregulation and nitrate inhibition of nodule formation in soybean cv. Enrei and its nodulation mutants. J Exp. Bot. 44, 547-553.
Heckmann, A., Lombardo, F., Miwa, H., Perry, J., Bunnewell, S., Parniske, M., Wang, T. & Downie, J. (2006) Lotus japonicus nodulation requires two GRAS domain regulators, one of which is functionally conserved in a non-legume. Plant Physiol. 142, 1739-1750.
Herridge, D. F. (1982) Relative abundance of ureides and nitrate in plant tissues of soybean as a quantitative assay of nitrogen fixation. Plant Physiol. 70, 1-6.
Indrasumunar, A. (2007) ‘Molecular cloning and functional characterisation of soybean (Glycine max L.) Nod factor receptor genes’. Doctor of Philosophy thesis, School of Integrative Biology. University of Queensland.
Indrasumunar, A., Kereszt, A., Searle, I., Miyagi, M., Li, D., Nguyen, C., Men, A., Carroll, B. & Gresshoff, P. (2010) Inactivation of duplicated Nod-Factor Receptor 5 (NFR5) genes in recessive loss-of-function non-nodulation mutants of allotetraploid soybean (Glycine max L. Merr.). Plant Cell Physiol. 51, 201-214.
Ito, Y., Nakanomyo, I., Motose, H., Iwamoto, K., Sawa, S., Dohmae, N. & Fukuda, H. (2006) Dodeca-CLE peptides as suppressors of plant stem cell differentiation. Science 313, 842-845.
Jeudy, C., Ruffel, S., Freixes, S., Tillard, P., Santoni, A. L., Morel, S., Journet, E.-P., Due, G., Gojon, A., Lepetit, M. & Salon, C. (2010) Adaptation of Medicago truncatula to nitrogen limitation is modulated via local and systemic nodule developmental responses. New Phytol. 185, 817-828.
Kereszt, A., Li, D., Indrasumunar, A., Nguyen, C. D. T., Nontachaiyapoom, S., Kinkema, M. & Gresshoff, P. M. (2007) Agrobacterium rhizogenes-mediated transformation of soybean to study root biology. Nat. Protocols 2, 948-952.
Kondo, T., Sawa, S., Kinoshita, A., Mizuno, S., Kakimoto, T., Fukuda, H. & Sakagami, Y. (2006) A plant peptide encoded by CLV3 identified by in situ MALDI-TOF MS analysis. Science 313, 845.
Kosuta, S., Hazledine, S., Sun, J., Miwa, H., Morris, R. J., Downie, J. A. & Oldroyd, G. E. D. (2008) Differential and chaotic calcium signatures in the symbiosis signaling pathway of legumes. Proc. Natl. Acad. Sci. U.S.A. 105, 9823-9828.
Krusell, L., Madsen, L. H., Sato, S., Aubert, G. g., Genua, A., Szczyglowski, K., Duc, G. r., Kaneko, T., Tabata, S., Bruijnk, F. d., Pajuelo, E., Sandal, N. & Stougaard, J. (2002) Shoot control of root development and nodulation is mediated by a receptor-like kinase. Nature 420, 422-426.
Larkin, M. A., Blackshields, G., Brown, N. P., Chenna, R., McGettigan, P. A., McWilliam, H., Valentin, F., Wallace, I. M., Wilm, A. & Lopez, R. (2007) Clustal W and Clustal X version 2.0. Bioinformatics 23, 2947-2948.
Li, D., Kinkema, M. & Gresshoff, P. M. (2009) Autoregulation of nodulation (AON) in Pisum sativum (pea) involves signalling events associated with both nodule primordia development and nitrogen fixation. J. Plant Physiol. 166, 955-967.
Lin, Y.-H., Ferguson, B. J., Kereszt, A. & Gresshoff, P. M. (2010) Suppression of hypernodulation in soybean by a leaf-extracted, NARK- and Nod factor-dependent low molecular mass fraction. New Phytol. 185, 1074-1086.
Magori, S. & Kawaguchi, M. (2009) Long-distance control of nodulation: Molecules and models. Mol. Cells 27, 129-134.
Meixner, C., Ludwig-Müller, J., Miersch, O., Gresshoff, P., Staehelin, C. & Vierheilig, H. (2005) Lack of mycorrhizal autoregulation and phytohormonal changes in the supernodulating soybean mutant nts1007. Planta 222, 709-715.
Meixner, C., Vegvari, G., Ludwig-Muller, J., Gagnon, H., Steinkeliner, S., Staehelin, C., Gresshoff, P. & Vierheilig, H. (2007) Two defined alleles of the LRR receptor kinase GmNARK in supernodulating soybean govern differing autoregulation of mycorrhization. Physiol. Plant 130, 261-270.
Meng, L., Ruth, K. C., Fletcher, J. C. & Feldman, L. (2010) The roles of different CLE domains in Arabidopsis CLE polypeptide activity and functional specificity. Mol Plant 10.1093/mp/ssq021.
Middleton, P. H., Jakab, J., Penmetsa, R. V., Starker, C. G., Doll, J., Kalo, P., Prabhu, R., Marsh, J. F., Mitra, R. M., Kereszt, A., Dudas, B., VandenBosch, K., Long, S. R., Cook, D. R., Kiss, G. B. & Oldroyd, G. E. D. (2007) An ERF transcription factor in Medicago truncatula that is essential for Nod factor signal transduction. Plant Cell 19, 1221-1234.
Mortier, V., Den Herder, G., Whitford, R., Van de Velde, W., Rombauts, S., D'haeseleer, K., Holsters, M. & Goormachtig, S. (2010) CLE peptides control Medicago truncatula nodulation locally and systemically. Plant Physiol. 153, 222-237.
Murray, J. D., Karas, B. J., Sato, S., Tabata, S., Amyot, L. & Szczyglowski, K. (2007) A cytokinin perception mutant colonized by Rhizobium in the absence of nodule organogenesis. Science 315, 101-104.
Ni, J. & Clark, S. E. (2006) Evidence for functional conservation, sufficiency, and proteolytic processing of the CLAVATA3 CLE domain. Plant Physiol. 140, 726-733.
Nishimura, R., Hayashi, M., Wu, G. J., Kouchi, H., Imaizumi-Anraku, H., Murakami, Y., Kawasaki, S., Akao, S., Ohmori, M. & Nagasawa, M. (2002) HAR1 mediates systemic regulation of symbiotic organ development. Nature 420, 426-429.
Nontachaiyapoom, S., Scott, P. T., Men, A. E., Kinkema, M., Schenk, P. M. & Gresshoff, P. M. (2007) Promoters of orthologous Glycine max and Lotus japonicus nodulation autoregulation genes interchangeably drive phloem-specific expression in transgenic plants. Mol. Plant-Microbe Interact. 20, 769-780.
Norris, S., Meyer, S. & Callis, J. (1993) The intron of Arabidopsis thaliana polyubiquitin genes is conserved in location and is a quantitative determinant of chimeric gene expression. Plant Mol. Biol. 21, 895-906.
Ogawa, M., Shinohara, H., Sakagami, Y. & Matsubayashi, Y. (2008) Arabidopsis CLV3 peptide directly binds CLV1 ectodomain. Science 319, 294.
Ohyama, K., Shinohara, H., Ogawa-Ohnishi, M. & Matsubayashi, Y. (2009) A glycopeptide regulating stem cell fate in Arabidopsis thaliana. Nat. Chem. Biol. 5, 578-580.
Oka-Kira, E. & Kawaguchi, M. (2006) Long-distance signaling to control root nodule number. Curr. Opin. Plant Biol. 9, 1-7.
Okamoto, S., Ohnishi, E., Sato, S., Takahashi, H., Nakazono, M., Tabata, S. & Kawaguchi, M. (2009) Nod factor, nitrate-induced CLE genes that drive systemic regulation of nodulation. Plant Cell Physiol. 50, 67-77.
Oldroyd, G. E. & Downie, J. A. (2008) Coordinating nodule morphogenesis with rhizobial infection in legumes. Annu. Rev. Plant Biol. 59, 519-546.
Oldroyd, G. E. D. & Downie, J. A. (2006) Nuclear calcium changes at the core of symbiosis signalling. Curr. Opin. Plant Biol. 9, 351-357.
Olsson, J., Nakao, P., Bohlool, B. & Gresshoff, P. (1989) Lack of systemic suppression of nodulation in split root systems of supernodulating soybean (Glycine max [L.] Merr.) mutants. Plant Physiol. 90, 1347-1352.
Ramakers, C., Ruijter, J. M., Deprez, R. H. L. & Moorman, A. F. M. (2003) Assumption-free analysis of quantitative real-time polymerase chain reaction (PCR) data. Neurosci. Lett 339, 62-66.
Sagan M. & Duc G. (1996). Sym28 and Sym29, two new genes involved In regulation of nodulation in pea (Pisum sativum L). Symbiosis, 20, 229-245.
Salamov, A. & Solovyev, V. (2000) Ab initio gene finding in Drosophila genomic DNA. Genome Res. 10, 516-522.
Schauser, L., Roussis, A., Stiller, J. & Stougaard, J. (1999) A plant regulator controlling development of symbiotic root nodules. Nature 402, 191-194.
Schmutz, J., Cannon, S. B., Schlueter, J., Ma, J., Mitros, T., Nelson, W., Hyten, D. L., Song, Q., Thelen, J. J., Cheng, J., Xu, D., Hellsten, U., May, G. D., Yu, Y., Sakurai, T., Umezawa, T., Bhattacharyya, M. K., Sandhu, D., Valliyodan, B., Lindquist, E., Peto, M., Grant, D., Shu, S., Goodstein, D., Barry, K., Futrell-Griggs, M., Abernathy, B., Du, J., Tian, Z., Zhu, L., Gill, N., Joshi, T., Libault, M., Sethuraman, A., Zhang, X.-C., Shinozaki, K., Nguyen, H. T., Wing, R. A., Cregan, P., Specht, J., Grimwood, J., Rokhsar, D., Stacey, G., Shoemaker, R. C. & Jackson, S. A. (2010) Genome sequence of the paleopolyploid soybean (Glycine max (L.) Merr.). Nature 463 178-183.
Schnabel, E., Journet, E.-P., Carvalho-Niebel, F. d., Duc, G. & Frugoli, J. (2005) The Medicago truncatula SUNN gene encodes a CLV1-like leucine-rich repeat receptor kinase that regulates nodule number and root length. Plant Mol. Biol. 58, 809-822.
Searle, I. R., Men, A. E., Laniya, T. S., Buzas, D. M., Iturbe-Ormaetxe, I., Carroll, B. J. & Gresshoff, P. M. (2003) Long-distance signaling in nodulation directed by a CLAVATA1-like receptor kinase. Science 299, 109-112.
Sulieman, S., Fischinger, S., Gresshoff, P. & Schulze, J. (2010) Asparagine as a major factor in the N-feedback regulation of N₂ fixation in Medicago truncatula. Physiol. Plant 10.1111/j.1399-3054.2010.01380.x.
Tanaka, A., Fujita, K. & Terasawa, H. (1985) Growth and dinitrogen fixation of soybean root system affected by partial exposure to nitrate. Soil Sci. Plant Nutr. 31, 637-645.
Tirichine, L., Sandal, N., Madsen, L. H., Radutoiu, S., Albrektsen, A. S., Sato, S., Asamizu, E., Tabata, S. & Stougaard, J. (2007) A gain-of-function mutation in a cytokinin receptor triggers spontaneous root nodule organogenesis. Science 315, 104-107.
Vernie, T., Moreau, S., de Billy, F., Plet, J., Combier, J. P., Rogers, C., Oldroyd, G., Frugier, F., Niebel, A. & Gamas, P. (2008) EFD Is an ERF Transcription Factor Involved in the Control of Nodule Number and Differentiation in Medicago truncatula. Plant Cell 20, 2696-2713.
Wang, J., Lee, C., Replogle, A., Joshi, S., Korkin, D., Hussey, R., Baum, T., Davis, E., Wang, X. & Mitchum, M. (2010) Dual roles for the variable domain in protein trafficking and host-specific recognition of Heterodera glycines CLE effector proteins. New Phytol. 10.1111/j.1469-8137.2010.03300.x.
Wesley, S., Helliwell, C., Smith, N., Wang, M., Rouse, D., Liu, Q., Gooding, P., Singh, S., Abbott, D., Stoutjesdijk, P., Robinson, S., Gleave, A., Green, A. & Waterhouse, P. (2001) Construct design for efficient, effective and high-throughput gene silencing in plants. Plant J. 27, 581-590.

1-33. (canceled)

34. An isolated protein which comprises an amino acid sequence of a CLAVATA3/ESR-related (CLE) protein that is responsive to Rhizobiales inoculation, nitrate or ammonium in a soybean plant, wherein the amino acid sequence is set forth in SEQ ID NO:5, SEQ ID NO:6 or SEQ ID NO:7.

35. A variant of the isolated protein of claim 34 selected from the group consisting of: (i) a variant comprising an amino acid sequence which has at least 80% sequence identity to an amino acid sequence set forth in SEQ ID NO:6; (ii) a variant which comprises an amino acid sequence having at least 90% sequence identity to an amino acid sequence set forth in SEQ ID NO:5 or SEQ ID NO:7; and (iii) a variant comprising an amino acid sequence having at least 95% sequence identity to an amino acid sequence set forth in SEQ ID NO:5, SEQ ID NO:6 or SEQ ID NO:7.

36. The variant of claim 35, which comprises one or more amino acid deletions or substitutions in an amino acid sequence thereof according to RLX₁P₁X₂GP₂DX₃X₄HX₅ (SEQ ID NO:4) wherein X₁=serine or alanine; X₂=glycine or glutamate; X₃=proline or glutamine; X₄=histidine, lysine or glutamine; and X₅=histidine or asparagine, with the proviso that when X₁ is serine, X₄ is lysine and X₅ is histidine, said one or more amino acid deletions or substitutions selected from the group consisting of (i) R is absent or is substituted by another amino acid; (ii) X₁ is absent or is substituted by an amino acid other than A or S; (iii) P₁ is absent or is substituted by another amino acid; (iv) P₂ is absent or is substituted by another amino acid; (v) G is absent or substituted by an amino acid other than alanine; (vi) D is absent or is substituted by another amino acid; (vii) H is absent or is substituted by another amino acid; and (viii) X₅ is absent or is substituted by an amino acid other than H or N.

37. A biologically active fragment of the isolated protein of claim 34, which comprises at least twelve (12) contiguous amino acids of SEQ ID NO:5, SEQ ID NO: 6 or SEQ ID NO:7.

38. The biologically active fragment of claim 37, which comprises an amino acid sequence according to SEQ ID NO:1, SEQ ID NO:2 or SEQ ID NO:3.

39. An antibody or antibody fragment which binds and/or is raised against the isolated protein of claim 34.

40. An isolated nucleic acid comprising a nucleotide sequence selected from the group consisting of: (a) a nucleotide sequence encoding an isolated protein which comprises an amino acid sequence of a CLAVATA3/ESR-related (CLE) protein that is responsive to Rhizobiales inoculation, nitrate or ammonium in a soybean plant, wherein the amino acid sequence is set forth in SEQ ID NO:5, SEQ ID NO:6 or SEQ ID NO:7; (b) a nucleotide sequence encoding an amino acid sequence which has at least 80% sequence identity to an amino acid sequence set forth in SEQ ID NO:6 or an amino acid sequence having at least 90% sequence identity to an amino acid sequence set forth SEQ ID NO:5 or SEQ ID NO:7; and (c) a biologically active fragment of the isolated protein in (a) which comprises at least twelve (12) contiguous amino acids of SEQ ID NO:5, SEQ ID NO: 6 or SEQ ID NO:7.

41. The isolated nucleic acid of claim 40, comprising a nucleotide sequence selected from the group consisting of: a nucleotide sequence set forth in SEQ ID NO: 11; a nucleotide sequence set forth in SEQ ID NO: 12; and a nucleotide sequence set forth in of SEQ ID NO: 13.

42. An isolated nucleic acid comprising a promoter, or promoter-active fragment of a CLE gene that comprises a nucleotide sequence set forth in any one of SEQ ID NOS:14-16.

43. A genetic construct comprising the isolated nucleic acid of claim 42 operably linked to a heterologous nucleotide sequence.

44. A genetic construct comprising the isolated nucleic acid of claim 41 operably linked or connected to one or more regulatory sequences in an expression vector.

45. A genetically-modified leguminous crop plant, or one or more cells or tissues of said plant, comprising and expressing: the genetic construct of claim 44, wherein the genetically-modified plant displays relatively improved, enhanced and/or otherwise facilitated nodulation and/or nitrogen fixation compared to a non-genetically modified leguminous crop plant, or one or more cells or tissues of said plant.

46. The genetically-modified leguminous crop plant, plant cell or tissue of claim 45, which is a soybean or common bean plant, cell or tissue.

47. A method of producing a genetically-modified leguminous crop plant, cell or tissue including the step of introducing the genetic construct of claim 44 into a cell or tissue of a leguminous crop plant; to thereby produce said genetically-modified leguminous crop plant, plant cell or tissue, wherein the genetically-modified leguminous crop plant, cell or tissue displays relatively improved, enhanced and/or otherwise facilitated nodulation and/or nitrogen fixation compared to said a leguminous crop plant.

48. The method of claim 47, wherein said genetically-modified leguminous crop plant is soybean or common bean.

49. A method of breeding a leguminous crop plant, said method including the step of crossing parent leguminous crop plants to produce a progeny leguminous crop plant having a desired trait associated with CLE protein-regulated nodulation and/or nitrogen fixation, wherein at least one of the parent leguminous crop plants is selected as having the desired trait associated with CLE protein-regulated nodulation and/or nitrogen fixation, wherein the CLE protein is according to claim 34, wherein the progeny plant, cell or tissue displays relatively improved, enhanced and/or otherwise facilitated nodulation and/or nitrogen fixation compared to one or more of the parent plants.

50. The method of claim 49, wherein the leguminous crop plant is soybean or common bean.


SEQUENCE LISTING (28 sequences)

SEQ ID NO: 1 - protein, 12 aa, Glycine max
RLAPEGPDPHHN

SEQ ID NO: 2 - protein, 12 aa, Glycine max
RLSPGGPDQKHH

SEQ ID NO: 3 - protein, 12 aa, Glycine max
RLAPGGPDPQHN

SEQ ID NO: 4 - protein, 12 aa, Artificial sequence
  Consensus sequence
RLXPXGPDXXHX

SEQ ID NO: 5 - protein, 95 aa, Glycine max
MANATMATRVSILIALIILSTFFMTLQASNLHGHPFIRENNIADSHHFLHKYLDDLSKHIHVQDADDAPHKDGNTHRLAPEGPDPHHNFATPPRN

SEQ ID NO: 6 - protein, 87 aa, Glycine max
MLEALGEMANAKQVLCLILLVLLFSKLESRSLEAFIEGKKTLAKGCSRELIEKSQLLKASFAKATTRFTNPRVSKRLSPGGPDQKHH

SEQ ID NO: 7 - protein, 93 aa, Glycine max
MGNTSATLVPILALIMFSTFFMTLQARSLHGHNPLFAHKKVVDIQNFLHKSGIHLSKRVRIPFGDDLPLAPADRLAPGGPDPQHNVRAPPRKP

SEQ ID NO: 8 - DNA, 36 bp, Glycine max
aggctcgcaccagagggaccagatcctcatcataat

SEQ ID NO: 9 - DNA, 36 bp, Glycine max
agactaagccctggaggacctgatcaaaaacatcac

SEQ ID NO: 10 - DNA, 36 bp, Glycine max
agacttgcaccaggaggaccagatcctcagcataat

SEQ ID NO: 11 - DNA, 288 bp, Glycine max
atggcaaatgcaacaatggctactcgagtgtctatactaattgcactaatcatcctctccaccttcttcatgactttgcaggctagtaatctccatggccatcccttcattcgtgaaaataatattgcagatagccatcactttcttcacaaatatttagacgacctatcaaagcatatacatgttcaagatgctgatgatgcccctcacaaagatggaaatacgcataggctcgcaccagagggaccagatcctcatcataattttgcaacaccaccaagaaactaa

SEQ ID NO: 12 - DNA, 264 bp, Glycine max
atgcttgaagccttgggggaaatggccaacgcaaagcaagtactatgcctaattttgttggtgcttttattttccaagcttgaaagtcgttctcttgaggcattcatagagggaaagaagaccctagccaaaggttgttcacgagaattgattgaaaaatcacagctcctgaaggcaagttttgcaaaagcaacaacacgttttacaaatcctagagtgtccaaaagactaagccctggaggacctgatcaaaaacatcactaa

SEQ ID NO: 13 - DNA, 282 bp, Glycine max
atgggaaatacaagtgcaaccctagtgcccatacttgccctaatcatgttctctacattcttcatgactttgcaagctcgtagtctccatggccacaatccattattcgctcacaaaaaagttgttgacatccaaaactttctccacaaatcgggtattcacctatcaaagcgtgtgcgtattccatttggtgatgatctcccactagcacctgcagatagacttgcaccaggaggaccagatcctcagcataatgtgagagctccaccacgcaagccatag

SEQ ID NO: 14 - DNA, 1564 bp, Glycine max
agggatagtcaagcttctacagatgccaaagagttaattatcatttccaaattcacaggttttcccccttacagagagaaaaagagtaatgatgcataatgttgggattttcacatagtgtttgctcatcatacgtacatgcataattatatgaataaatatggtttattttccgaaaaaaatggtttgttttattttataagtgattaaaaataaataaatataattatgtatagtcattgtttgtgcttgtgaaattaatggttaaatatgtttaacaagtaattaatagtgtcataagattattaaaatatctaatgttaattaattaacttaattttttataagtgattaagagtaaataaatactagtaggtgtacgtatataattatcaatttgtagctatttgtaattaaagattaaatatgtcttacaagacattcaccaaaaaaaaaaaatgtcttacaagtaattaatagtgtcataagattattgaaattatctatatttatatggcatttatggctaattgtgccgagtagaggtgggatccagcaagaccttgataattttcatacaaaaaagaatcccgaaacctaacattacagcaagtagtaaaatttcgacgatctgggttcaagcagttatgcatctacgtacgtattgactctttatgataaaaatccctcattcatctgaagagttaagttggtgattgatattatatatggcagagttttatgattaatttagttatgactttgtttcttatccctcagaatttcttttaatagaaacataataattaagtacatcatcatatatatgtttcgtttatactccaaaccttaaggattcttatgttaggataggaaaaatatatagggagaggagtggtaaagttattcagagagtaatcttaaaaaatttattccgaatctgaagactcttattaaattgggaaaactccttttcatttactggaaattgaagagaacccctcccctattagaccctctcgatcatctctcacaataaaacttctttctcacctgacttatatatgtatatatataaaccttttatgagcattcaacactaaccgtttagcctctagagagttaattaattattaagtagactgcgacttgtatttcattaaagccatgtttcttattttgcccatatttttattttatattaaattcataataaaataacaattttattttttatttttgacacttatttttaatcttgataaattagtaaattttatatttattattagttaagactaaaataaaatttattaatttattaatgaccaaacataagatttattaatttatcaaagattaaaataaaatataaaaaaaattattttattacagttcaaacataaaaaatttattaaaatataaacataaaaattaaatatttattatcaatcaaaaccatatttaatcctataaataaataatgttgatagtatgcttccacacatccaacctcaagtagaccttgttcaatattgattcagactttacatttcaccgcatg

SEQ ID NO: 15 - DNA, 1565 bp, Glycine max
taattcaatgagttatggatgaattgaattttttttcgatattataagatcatatatgaattaatgaacttttttttaccgcacacattaatggactatattaccattctagttaaattgttactaactagagtgtaaaaatgtctttagtaattgtctctctattttttttttgtaagacatgcattgtaactattaacaagagcaagttttaacaatcaacattttttatataaaaaaatgttaggacatgacattttttttggttgtggaacaccattccggtttcatttattatttcctctttttggcttaccaatcacttacattattatttgcagaatatattttctcatagattacttttggcttaatgaatctcgaagttgtttatattgaaaatttccatctgaagtcagactctttttcggttcgccacatttgtttcgatgagccttacgtcgtgctgttttcgtcctctcatattcggaaagcgccactatccgcaataataattagtgcacttacaaacgccactatccgcaatagtaattaataataaaccttcaataataatcttactcctttaaagtaataataataagaagaagaataatttgttaaaaaaactaacaattaatattttagaatacatgggcgtgcgcacatatgggtgcttataataaattatgaaatttctatgaatcataatttacacttttattttgaactacatggatctcttactttaaaagagtaaaatctaaaaatataaatgagttatgagaattagtcgatttttcaatcattatttcaatagttttgtaagttagaacaattatgttttcacaactttccaattttttttatcaccaatattaattattagtttttgttaaaatgttagtgagaaattcaaacgccaaacgatttttttcctccttctccctttaatctttcaactctcatttccaactactaaattagtcttataacttctttataatttttcacgtgtgttgagaactaggaatttaattagaagaacaccaaaaaaaatagttgttgaatggcctttggaacacctataaggaaaagctttgatcggaatatataaccttttagactttgaaaggtcttctagtcctagttctcattgaaagtggacagctcaaatctacatttatattcaagtgagatattattatgcattttttaaaaagaagttgattaaaaaaatgcataccaatgccggaagtactagatgacaaatttgaatgcacattggatcacgacgtcatcttattcccatgctttttttcgtgtttggacctcacatgttagctaggtcttaaacatgaaataatactaattttatgttttaaaagaaattattaaactatggttcagtacattttaaagatagaacaatcaaagcattacataagtacatttaagaaaaaagtaaaatatttttattctataaaaaattgtaattaaaaagacaagcagaaaaattatttgtttcaatatttttactcgcatgccaaattttacttaagatg

SEQ ID NO: 16 - DNA, 1569 bp, Glycine max
ccactagttgttatgaaagtgattgacttcaagatatgtacaaatttttttatataatttggatgttattaattttaaaatttgattttgaaaatcgattctactcaataattttcttttctgttctcagtttaattctttaagttttaattggatatggcttatatagttttaaatagtcctcctgtaaattaacattcttacaattagtatttgtgactaataacccaaatctaaatgatacctaaggaaactagaatgtgatgcaaatcacaactctagaattactttcctcagttagctagtaatttgtcattttccaacattatacatgctccaattctgcgaaagtaaataatctttaagaattcacaagaaacccataagttaggagaggttttcttctatggaaacgacaaaagaaagaaatacaaaccaacaaaataaagaagaaaatataatcataaattctctatataaatttttttaagttattaagtgagatgtgtaattattttatattacattaaagatatcccccatgcaaaaaacttcttggaattgaaaggtggtaattagtaaatgcacaagttttctacgttgcattgaaattcaactttttattagaacacaataagtataataacgattaaattcaatagactagctacttagatatatctaaatgcccaatatattaagaaccggttaagggatatttgatttggttgttttctgctttcattttcattgtaaaataaaatatagtgatgaaaatgcgtttgttttaccttcctccttagctataaagtcttatttttagtattttcagttgaaatcagaaaccttattttgagtaaattgaaaacgtggtaataagaaatgtaattttaagtaaatctaaaaatacatttccttttgaaaacgtatttttagtgtttttatttcttgaaaatagaaaacaagaagtaaaaccaaacatgttttcaaaattctaatcttttaaaaatgaaaacagttttcagaaaataaaaatgcaaaccaaacacatcctaaatttttagggttaaaacgtactcttgattttataataaatctctaacaacgtattcttttgagctaatggattatttgacaatttacctttattattaaatctgcatattggtttactataagaccaaacctcaaagaatgcccatgcatgcattttactataaggcttaactattgaaatagagaggagatataaaataagtttgatggttgaaaaaaaaagtaaaaagagaaagtttgtgaatttgatttcttctactaataaaattaacatgctaacaaattaacatttgttgatcataaaaaaaacttttgaaataaagatatagttatattcaccagtgataggctgttgctaacctaaccacgtataaatagtgcttacaccttgtagtaaccttccaaaaactacccccaactccttgatcattccacaagccatctcaagcttatatataattgttctcactttatatcaccttgttcaattctaagacttacatcacgcatatg

SEQ ID NO: 17 - DNA, 27 bp, Artificial sequence
  Synthetic primer
gcgcgaattcccgcatggcaaatgcaa

SEQ ID NO: 18 - DNA, 37 bp, Artificial sequence
  Synthetic primer
gcgcggtacccgaaatctactccaaacaaagctactt

SEQ ID NO: 19 - DNA, 32 bp, Artificial sequence
  Synthetic primer
gcgcctcgagatgggaaatacaagtgcaaccc

SEQ ID NO: 20 - DNA, 29 bp, Artificial sequence
  Synthetic primer
gcgcggtaccactatggcttgcgtggtgg

SEQ ID NO: 21 - DNA, 28 bp, Artificial sequence
  Synthetic primer
gcgcctcgagatgcttgaagccttgggg

SEQ ID NO: 22 - DNA, 34 bp, Artificial sequence
  Synthetic primer
gcgcggtaccttagtgatgtttttgatcaggtcc

SEQ ID NO: 23 - DNA, 22 bp, Artificial sequence
  Synthetic primer
caaatgcaacaatggctactcg

SEQ ID NO: 24 - DNA, 22 bp, Artificial sequence
  Synthetic primer
gccatggagattactagcctgc

SEQ ID NO: 25 - DNA, 21 bp, Artificial sequence
  Synthetic primer
ggccacaatccattattcgct

SEQ ID NO: 26 - DNA, 21 bp, Artificial sequence
  Synthetic primer
acgcacacgctttgataggtg

SEQ ID NO: 27 - DNA, 21 bp, Artificial sequence
  Synthetic primer
gccaaaggttgttcacgagaa

SEQ ID NO: 28 - DNA, 21 bp, Artificial sequence
  Synthetic primer
gcaaaacttgccttcaggagc